(19) United States
(12) Patent Application Publication
(10) Pub. No.: US 2012/0129794 A1
(43) Pub. Date: May 24, 2012

(54) APPARATI, METHODS, AND COMPOSITIONS FOR UNIVERSAL MICROBIAL DIAGNOSIS, DETECTION, QUANTIFICATION, AND SPECIMEN-TARGETED THERAPY

(76) Inventors: Scot E. Dowd, Shallowater, TX (US); Randall D. Wolcott, Lubbock, TX (US); John P. Kennedy, Pooler, GA (US)

(21) Appl. No.: 13/386,720

(22) PCT Filed: Jul. 26, 2010
(86) PCT No.: PCT/US2010/002099

§ 371 Date: Jan. 24, 2012

Related U.S. Application Data
(60) Provisional application No. 61/271,675, filed on Jul. 24, 2009.
(60) Provisional application No. 61/278,054, filed on Oct. 2, 2009.
(60) Provisional application No. 61/399,915, filed on Jul. 20, 2010.

(51) Int. Cl. A61K 31/70; C12Q 1/70; A61P 31/00; C12Q 1/68

(52) U.S. Cl. 514/24; 435/6.15; 435/5; 435/6.12

(57) ABSTRACT

Microbial ecology of a specimen is evaluated using an approach (Level I) that utilizes nucleic acid amplification with specific gene primers that will identify panels of microorganisms and antibiotic-resistance factors generating a diagnostic report (optionally with quantification of each microorganism or antibiotic-resistance factor) and an approach (Level II) that utilizes universal or semi-universal primers to amplify conserved genes at a general or specific taxonomic level that are tagged specimen specifically using a genetic or chemical marker that is specific to the specimen from which it was derived, then sequencing the amplified products with highly-parallel, high-throughput technology to provide comprehensive sequences of the microbial population in the specimen followed by analysis of this sequence information and specific targeted information from Level I and/or Level II to generate a comprehensive analysis, interpretation, and/or diagnostic report.

### FIELD OF THE INVENTION

The purpose is to provide apparati, methods, and compositions for diagnosis of infectious disease, including identification of a plurality of bacteria, fungi, helminths, protozoa, and/or viruses in a complex specimen collected from a subject suspected of being infected, and specimen-targeted therapy for an infected subject. The present invention is directed to apparati, methods, and compositions for use with molecular methodologies for microbial detection and quantification. Further described are bioinformatics or computational methods that utilize the microbial detection and quantification, including antibiotic resistance and sensitivity profiles, that can guide a personalized therapeutic regimen, which is not limited to systemic, implanted, and/or topical treatments, including antibiotic, probiotic, host supportive, and antibiofilm treatments.

The invention also relates to apparati, methods, and compositions for quantitative testing of a specimen for bacterial, fungal, helminthal, protozoan, and/or viral microorganisms concurrently. Alternately, the invention relates to apparati, methods, and compositions for testing of a specimen from a subject to detect, quantitate, and/or monitor microbial diversity in a comprehensive manner with the utilization of novel computational or bioinformatics approaches to process information, and provide interpretive findings that guide therapy.

### BACKGROUND OF THE INVENTION

The current embodiments were developed to characterize the microbial ecology of any type of environment and specimen and as a universal microbial pathogen diagnostic to allow for patient-specific treatment of infections and microbial ecology health. Research on the microbial diversity of every environmental system such as the gastrointestinal tract of animals and humans, chronic or biofilm infections of tissues, microbial diversity in air, water, soil, deep-sea vents, within plants, and other higher life forms, is surprisingly scarce. Even though it is well understood that bacteria, fungi, helminths, protozoa, and viruses in every environment are vital components that contribute to a subjects' or ecosystems' health and well-being. The bacterial populations that reside in the gut of humans for instance are diverse and numerous; intestinal populations often exceed 10¹¹ CFU/gram feces. The majority of these bacteria are vital to the maintenance of subject's health and it is expected that even minor perturbations in these populations may cause dramatic shifts that can affect the subject's state of health. These beneficial health effects relate to the ability of these intestinal bacterial populations to supply vital nutrients, convert metabolites and beneficially interact with host cells. Information on microbial diversity within the gastrointestinal tract of humans has increased in recent years as a result of 16S rRNA or rDNA-based analyses, while similar data on the microbiomes of livestock and every other polymicrobial environment is remained lacking.

The primary reasons for the lack of knowledge regarding the composition of the microbiome of both environmental and clinical specimens relates to the difficulty and expense of methods used to evaluate these populations. Traditionally culture-based methods have been used to identify and enumerate commensal members of the ruminal and intestinal flora. Culture-based methods are extremely time-consuming. Further, to date researchers have only been able to culture approximately 1%-5% of the bacteria in the gut. Similar statistics are realized for any type of environment. Thus, culture-based methods are extremely biased in their evaluation of microbial diversity, tending to overestimate the importance of bacterial species such as Escherichia coli that easily grow on an agar surface. Thus, the utility of a diagnostic or microbial ecology assay that is able to evaluate most if not the majority of bacteria, fungi, helminths, protozoa, and viruses in a given specimen would prove immensely valuable to the general healthcare industry, including medicine. PCR has become a modern solution to detecting specific microorganisms and pathogens; however, PCR is specific to a given organism and cannot detect or in any way characterize novel, new or unknown microorganisms that may be present in a specimen. Described here is the first universal pathogen diagnostic approach and methods to provide interpretations of complex diagnostic results leading to patient-specific treatments including infections that are polymicrobial (multiple organisms) in nature.

Although molecular approaches may also introduce their own forms of bias, such as the ability to detect both viable, viable but non-culturable, and non-viable bacteria, they currently provide the most powerful tools available for elucidating the diversity of a the microbiome of any environment. The use of massively paralleled sequencing technologies, such as the embodiments disclosed herein, combined with molecular methods has proven exceptionally valuable for evaluating the microbiomes of subjects. Further, in the present embodiments we utilized a novel tag-encoded bacterial diversity amplification method that uses massively parallel sequencing or pyrosequencing techniques to determine the diversity within the intestinal microbiota. This method makes evaluation of the microbiome of any infections both comprehensive and cost effective. This method utilizes universal primers combined with “alien DNA tags or barcodes” to individually label a given specimen allowing downstream high-throughput sequencing and bioinformatic monitoring. This method may be combined with multiple individual targeted and universal polymerase chain reactions as a unified microbiome characterization and diagnostic system. Combined with software developed to process and analyze this microbiome data, the complete system represents a comprehensive and highly novel method for evaluating the microbiome of any clinical specimen.

All cutaneous lesions that are classified as chronic wounds possess surface associated bacteria, regardless of host impairments. Clearly, any host factors that impair healing must be managed, but host factors are not universal impediments and vary from patient to patient. Only microbial bioburden is present in every non-healing cutaneous wound, making microbial bioburden management a universal therapeutic strategy.

Indeed, most chronic wounds show an incredible diversity of bacterial and fungal species, and their community structures, combinations and synergies seem infinite. To simplify this concept, bioinformatics analyses of wound biodiversity data has been used by the present embodiments to identify dozens of co-occurring populations of microorganisms, termed functional equivalent pathogroups (FEPs), which appear to form common and somewhat recurring groups in chronic wounds. This high diversity, along with biofilm's intrinsic properties of resistance to antibiotics, biocides and host immunity, has made wound bioburden an increasingly appreciated potential universal barrier to healing chronic wounds.

Historically targeting the microorganisms that comprise a particular biofilm has been very difficult due to the lack of sufficiently comprehensive clinical diagnostic tools. There is a need to be able to diagnose these polymicrobial infections to enable patient-specific therapies to treat them. Clinical cultures (agar-based cultivation methods) are the current state of the art clinical pathogen diagnostic tools available for evaluating wound bioburden. However, within research and academia, it is well understood that most bacteria grow poorly or not at all in common clinical cultures (i.e., anaerobes, yeast, biofilm phenotype which are viable but non-culturable), and multiple species in biofilm phenotype remain difficult to diagnose in an economical manner using routine clinical culture.

Agar-based cultures are traditionally a method designed through pure culture to try to find the “one organism” causing an infection (i.e., Koch's postulates). The properties of clinical cultures that render them most irrelevant is the selection bias for microorganisms actually capable of growing easily in artificial laboratory media, and the fact that the vast majority of bacteria that have been scientifically identified in human infections, especially chronic infections, cannot grow in routine clinical cultures. Clinical culture methods have the advantage of providing resistance and sensitivity information, but these sensitivities are limited in their utility in chronic infections because in such specimens, bacteria and yeast exists mainly in polymicrobial communities. Further, culture sensitivities obtained from cultivation methods are relevant only to planktonic phenotype and do not account for the phenotypical differences expressed by bacterial biofilms. Moreover, clinical cultures provide information on “only those few” bacteria that can be propagated efficiently in the laboratory. There are many other significant limitations related to the use of clinical culture methods, which have been reviewed in more detail throughout the scientific literature. One factor that led to the development of the embodiments was to overcome the obvious erroneous reports from clinical specimens that returned as “no growth.” Simply stated, a diagnostic tool that returns a negative result, when there are obvious clinical signs of infection, provided no utility or direction.

Further, the inability to correctly assess specimen bacteria and fungi may have contributed to the current recommendations for limited and empiric antibiotic and biocide use in chronic infections (e.g., wound specimens, ENT specimens, UGT specimens and UTI specimens). The evidence disclosed herein, support that by specifically targeting these polymicrobial infections, identified by the embodiments with treatment options guided by the embodiments, outcomes are decidedly improved. Therefore, there is a significant medical need for accurate microbial diagnostic tools and bioinformatic tools to interpret the complex results that result from such comprehensive analysis.

Originally, the inventors viewed these chronic infections as comprised mainly of “known” pathogens such as Staphylococcus aureus, Pseudomonas aeruginosa, etc. that predominated over minor populations, which at the time were considered as contaminants. This view was fostered by data from agar cultures, which yielded a limited but readily manageable number of different bacterial species for a classically trained clinician. However, the highly variable clinical responses to treatments based on those diagnostics led to significant ambiguity regarding the value of such treatments. Subsequently, molecular methods based surveys of chronic infections including venous leg ulcers, decubitus ulcers, diabetic foot ulcers, non-healing surgical wounds, and ENT specimens were executed by the inventors. These surveys demonstrated a new microbial reality, that most chronic infections are propagated with polymicrobial communities of composed various classes including bacteria, fungi, helminths, protozoa, and viruses, and typically mixtures of more than one microorganism.

In the clinical setting, these new comprehensive molecular diagnostics have the ability to define and monitor this microbial reality in each specific specimen. Each infection, acute or chronic, can benefit from diagnostics and personalized treatment. Although no approved clinical strategies currently exist to directly attack the synergies or other quorum sensing activities within chronic infections, by targeting these defenses, the inventors demonstrate that other more traditional treatments such as selective biocides and antibiotics are more effective. The advantages of the embodiments disclosed herein are multifaceted, yielding very rapid, specific, sensitive, quantitative, comprehensive results with little selection bias that provide for patient-specific and specimen-targeted therapeutic strategies that result in unexpectedly improved clinical outcomes.

### SUMMARY OF THE INVENTION

Accurate, rapid, sensitive, and comprehensive microbial detection and quantification able to fully define any type of infection including polymicrobial infections can have a dramatic impact on appropriate treatment and subsequent outcomes in the practice of medicine as well as the informative study of microbial systems within bodily flora that are not currently in a pathogenic state. By way of example, the microbiota of an animal or human intestinal tract plays important roles in overall health, productivity and well being. To date, there remains a scarcity of information on the microbial diversity in all the potential environments on our planet and indeed the universe. Enhancing the efficiency of the intestinal and rumen populations, for example can dramatically improve the productivity of this segment of our food industry. The primary reason for this lack of data relates to the expense of methods needed to generate such data. Therefore, one aim of the present embodiments is to provide apparati, compositions, and methods that result in a more accurate, rapid and sensitive microbial diagnosis, detection and quantification that can dramatically impact the practice of medicine and animal research alike. The inventors have developed a tag-encoded FLX 16S or 18S rRNA, or 16S or 18S rDNA amplicon pyrosequencing (TEFAP) approach that is able to perform microbial diversity analyses of any type of environment or clinical specimen. bTEFAP is the bacterial version of this method. Due to the novelty of the embodiments, a never before realized characterization of the microbial diversity of any environment becomes relatively inexpensive in terms of both time and labor. Due to the implementation of certain aspects of the embodiments including a novel tag priming methodology and an efficient clinical bioinformatics pipeline for use with microbial diagnostics in humans and animals, more accurate, complete and efficient approaches to evaluating, identifying, characterizing, determining ecological, pathogen and other factors, including defining specific treatments or therapeutics that effect enhancement or control of the microbiome present in a specimen, are made possible and economical. Ultimately, they empower the ability to provide subject or environment specific care or remediation.

The present invention provides apparati, methods, and compositions for accurate, rapid, and sensitive microbial detection, including identification and quantification.

An aspect of the present invention is directed to the utilization of microbial tag-encoded FLX amplicon pyrosequencing (mTEFAP) methods and compositions to perform microbial diversity analyses, pathogenic diagnosis, and relative quantification.

Another aspect of the present invention is directed to the development and utilization of a novel tag priming methods and compositions for use with microbial detection in any environment including humans and animals. In the preferred embodiment, the methods and compositions further comprise bioinformatics systems for analysis of data generated by the mTEFAP method.

Another aspect of the present invention is directed to the methods for diagnosing clinical pathogens which combines the utilization of polymerase chain reaction testing for rapid screening, followed by mTEFAP for comprehensive microbial population and microbial ecology evaluation, testing, or diagnostics all performed on a single specimen, or specimens collected the same day and analyzed by a high-throughput sequence analysis or bioinformatics or computational system.

A first embodiment is a method for detecting a plurality of different microorganisms in at least one specimen obtained from a subject, the method comprising in any order: sequencing a plurality of genetic materials in a specimen; wherein the genetic materials are selected from the group consisting of amplified templates, genomes, or metagenomes; wherein the presence of a sequence indicative of a genus, species, or strain of microorganism is sufficient to identify or quantify proportionally that microorganism among the plurality of different microorganisms in a specimen; and amplifying target polynucleotides in a specimen to quantify the total or individual number of the plurality of different microorganisms in a specimen.

A second embodiment is a method for detecting a plurality of different microorganisms in at least one specimen obtained from a subject, the method comprising in any order: amplifying target polynucleotides in a specimen to produce template nucleic acids; wherein the presence of a template indicative of a genus, species, or strain of microorganism is sufficient to identify or quantify the plurality of different microorganisms in a specimen; and sequencing a plurality of genetic materials in a specimen; wherein the genetic materials are selected from the group consisting of amplified templates, genomes, or metagenomes; wherein the presence of a sequence indicative of a genus, species, or strain of microorganism is sufficient to identify or quantify proportionally that microorganism among the plurality of different microorganisms in a specimen. Optionally, the method may further comprise, in any order, amplifying target polynucleotides in a specimen to quantify the total or individual number of the plurality of different microorganisms in a specimen.

For multiplex analysis according to any of the above, N different specimens are amplified in parallel reactions by tagging target polynucleotides of a first specimen with a first marker, tagging target polynucleotides of a second specimen with a second marker, and so on mutatis mutandis to tagging target polynucleotides of an Nth specimen with an Nth marker prior to amplifying or sequencing; a marker is found in an amplified or sequenced template nucleic acid; and the marker identifies the template nucleic acid as derived from a particular specimen. At least 10, at least 25, at least 50, at least 75, at least 100, or at least 250 different specimens may be amplified and/or sequenced in parallel reactions.

In accordance with any of the above methods, at least one, two, three, or four microbial genera, species, and/or strains detected at a proportion less than 1%, less than 2.5%, or less than 5% in the specimen may not be reported as detected or may be reported as not detected. A report comprising microbial genera, species, and/or strains detected (or not) may be prepared for a physician to guide antimicrobial treatment of the subject.

In accordance with any of the above methods, at least one, two, three, or four microbial genera, species, and/or strains may be detected at a number less than 10, less than 100, less than 1000, or less than 10,000 in the specimen may not be reported as detected or may be reported as not detected. A report comprising microbial genera, species, and/or strains detected (or not) may be prepared for a physician to guide antimicrobial treatment of the subject.

In accordance with any of the above methods, at least five, at least ten, at least 15, at least 20, at least 25, at least 30, at least 35, at least 40, at least 45, at least 50, 100, 1000 or 10,000 different microbial genera, species, and/or strains may be detected in a specimen. A report comprising microbial genera, species, and/or strains detected (or not) may be prepared for a physician to guide antimicrobial treatment of the subject.

In accordance with any of the above methods, the set of amplification primers may anneal to a single-copy gene sequence present in a genus, species, or strain of microorganism; or ribosomal gene sequence present in a single or multiple species of bacteria or yeast.

In accordance with any of the above methods, the set of amplification primers may anneal to a ribosomal (e.g., 16S) gene sequence present in a single or multiple species of bacteria.

In accordance with any of the above methods, the set of amplification primers may anneal to a ribosomal (e.g., 18S) gene sequence present in a single or multiple species of yeast.

In accordance with any of the above methods, amplification reactions may be performed using a nucleic acid amplifier instrument.

In accordance with any of the above methods, sequence reactions may be performed using a nucleic acid sequencer instrument.

In accordance with any of the above methods, at least five, at least ten, at least 15, at least 20, at least 25, at least 30, at least 35, at least 40, at least 45, or at least 50 different microbes may be detectable by amplification using specific primers for the following genera or their species: Pseudomonas, Corynebacterium, Staphylococcus, Serratia, Enterococcus, Streptococcus, Finegoldia, Anaerococcus, Escherichia, Pelomonas, Bacteroides, Fusobacterium, Prevotella, Acinetobacter, Proteus, Ralstonia, Haemophilus, Peptoniphilus, Peptostreptococcus, Veillonella, Porphyromonas, Klebsiella, Brevibacterium, Moraxella, Enterobacter, Stenotrophomonas, Morganella, Clostridium, Propionibacterium, Helicobacter, Citrobacter, Terrimonas, Candidatus, Parvimonas, Burkholderia, Fastidiosipila, Flavobacterium, Ruminococcus, Helcococcus, Roseateles, Turicibacter, Rhizobium, Mycoplasma, Conexibacter, Merismopedia, Salmonella, Sporanaerobacter, Actinomyces, Neisseria, Anabaena, Granulicatella, Hydrocarboniphaga, Raoultella, Dermabacter, Curvibacter, Macrococcus; Lactobacillus, Arcanobacterium, Allobaculum, Providencia, Brevibacterium, Alkalibacterium, Eubacterium, and Achromobacter. A report comprising bacterial genera detected (or not) may be prepared for a physician to guide antimicrobial treatment of the subject.

A third embodiment is a method for detecting a plurality of different microorganisms in at least one wound specimen obtained from a subject, the method comprising: amplifying target polynucleotides in a specimen with a set of primer oligonucleotides to produce template nucleic acids, wherein the presence of a template indicative of a specific taxonomic designation of genus is sufficient to identify or quantify that microorganism in a specimen; wherein the set of primers are able to detect all of Pseudomonas, Corynebacterium, Staphylococcus, Serratia, Enterococcus, Streptococcus, Finegoldia, and Anaerococcus; and wherein the set of primers are further able to detect one or more of the following sets: Set A (i.e., Escherichia, Pelomonas, Bacteroides, Fusobacterium, Prevotella, Acinetobacter, Proteus, and Ralstonia); or Set B (i.e., Haemophilus, Peptoniphilus, Peptostreptococcus, Veillonella, Porphyromonas, Klebsiella, Brevibacterium, and Moraxella); or Set C (i.e., Enterobacter, Stenotrophomonas, Morganella, Clostridium, Propionibacterium, Helicobacter, Citrobacter, and Terrimonas); Set D (i.e., Candidatus, Parvimonas, Burkholderia, Fastidiosipila, Flavobacterium, Ruminococcus, Helcococcus, and Roseateles); or Set E (i.e., Turicibacter, Rhizobium, Mycoplasma, Conexibacter, Merismopedia, Salmonella, Sporanaerobacter, and Actinomyces); or Set F (i.e., Neisseria, Anabaena, Granulicatella, Hydrocarboniphaga, Raoultella, Dermabacter, Curvibacter, and Macrococcus); or Set G (i.e., Lactobacillus, Arcanobacterium, Allobaculum, Providencia, Brevibacterium, Alkalibacterium, Eubacterium, and Achromobacter). A report comprising bacterial genera detected (or not) may be prepared for a physician to guide antimicrobial treatment of the subject.

A fourth embodiment is a method for detecting a plurality of different microorganisms in at least one respiratory specimen obtained from a subject, the method comprising: amplifying target polynucleotides in a specimen with a set of primer oligonucleotides to produce template nucleic acids, wherein the presence of a template indicative of a specific taxonomic designation of genus is sufficient to identify or quantify that microorganism in a specimen; wherein the set of primers are able to detect all of Streptococcus pneumoniae, Haemophilus influenza, Moraxella catarrhalis, Staphylococcus aureus, methicillin resistant staphylococcus, Streptococcus pyogenes, Streptococcus mitis, and Pseudomonas aeruginosa; and wherein the set of primers are further able to detect one or more of the following sets: Set A (i.e., Yeast spp., Candida albicans, Staphylococcus epidermidis, Staphylococcus haemolyticus, Fusobacterium spp., Eikenella corrodens, E. coli, and Klebsiella spp.); or Set B (i.e., Aspergillus spp., Haemophilus parainfluenzae, Bacteroides fragilis, Proprionibacterium spp., Corynebacterium spp., Turicella spp., Enterococcus spp., and Achromobacter spp.); or Set C (i.e., Citrobacter spp., Serratia spp., Proteus spp., Prevotella spp., Stenotrophomonas spp., Actinomyces spp., Peptostreptococcus spp., and Meningococcus spp.); Set D (i.e., Bacillus spp., Mycobacterium tuberculosis, Respiratory Syncytial Virus, Influenza A, Influenza B, Parainfluenza, Rhinovirus, and Adenovirus); or Set E (i.e., Metapneumovirus, Echo Virus, Coxsackie Virus, Herpes Virus, Corona Virus, Epstein Barr Virus, Cytomegalovirus, and Enterovirus); or Set F (i.e., Streptococcus algalactiae, Streptococcus mutans, Porphyromonas gingivalis, Streptococcus sanguinis, Veillonella spp., Bartonella spp., Mycobacterium avium, Mycobacterium bovis, and Mycoplasma pneumoniae); or Set G (i.e., Chlamydophila pneumoniae, Legionella spp., Enterobacter aerogenes, Enterobacter cloacae, Borrelia burgdorferi, Moraxella canis, Burkholderia spp., Eubacterium spp., and Treponema spp.). A report comprising bacterial genera detected (or not) may be prepared for a physician to guide antimicrobial treatment of the subject.

A fifth embodiment is a method for detecting a plurality of different microorganisms in at least one blood specimen obtained from a subject, the method comprising: amplifying target polynucleotides in a specimen with a set of primer oligonucleotides to produce template nucleic acids, wherein the presence of a template indicative of a specific taxonomic designation of genus is sufficient to identify or quantify that microorganism in a specimen; wherein the set of primers are able to detect all of Borrelia burgdorferi, Bartonella henselae, and Brachyspira hyodysenteriae; and wherein the set of primers are further able to detect one or more of the following sets: Set A (i.e., Coxiella burnetii, Leptospira biflexa, Mycoplasma fermentans, and Mycoplasma hyopharyngis); or Set B (i.e., any three of Borrelia afzelii, Borrelia garinii, Borrelia hermsii, Borrelia lonestari, and Borrelia parkeri) or Set C (i.e., Mycoplasma fermentans and Mycoplasma hyopharyngis); Set D (i.e., any four of Rickettsia rickettsii, Rickettsia akari, Rickettsia conorii, Rickettsia sibirica, Rickettsia australis, Rickettsia japonica, Rickettsia africae, Rickettsia prowazekii, and Rickettsia typhi); or Set E (i.e., any two of Anaplasma phagocytophila, Francisella tularensis, Brachyspira aalborgi, Ehrlichia chaffeensis, and Ehrlichia ewingii); or Set F (i.e., any two of Leptospira borgpetersenii, Leptospira interrogans, Leptospira kirschneri, and Leptospira wolbachii); or Set G (i.e., any two of Treponema denticola, Treponema carateum, Treponema pallidum, and Treponema pertenue). A report comprising bacterial genera detected (or not) may be prepared for a physician to guide antimicrobial treatment of the subject.

After in vitro diagnosis according to any of the above, the subject may be treated by a physician in accordance with the specific microorganisms that were detected (or not detected or present at below a detectable limit). For example, a method for treating a subject with an infection comprising detecting a plurality of different microorganisms in at least one specimen obtained from the subject, then administering a treatment regimen that is effective against at least one or multiple microorganisms that were detected. For this purpose, a report may be generated listing the plurality of microorganisms that were detected (or not) by their genus, species, and/or strain. Treatment may include at least one or multiple antibiotics, one or more antibiofilm agents, or both.

Alternately, the subject may be monitored for treatment efficacy by detecting a plurality of different microorganisms in at least one specimen obtained from the subject after initial treatment of the infection. For this purpose, a report may be generated listing the plurality of microorganisms that were detected (or not) by their genus, species, and/or strain.

The purpose of the invention is to provide apparati, methods, compositions, and workflows, or components thereof, devices and methods that improve the evaluation of microbial diversity in any environment, and further provide the ability to perform comprehensive microbial population characterization in a system that directs personalized treatments or remedies or enhancements, thereby these embodiments will make such treatments, remedies or enhancements specific to the subject or the environment and the delivery of the treatment more convenient, targeted, and effective. These combined benefits cascade to provide improved analytical efficiency, analytical accuracy, treatment efficiency, treatment accuracy, and treatment outcomes, while limiting errors in treatment, remedy, or enhancement.

### DESCRIPTION OF SPECIFIC EMBODIMENTS OF THE INVENTION

Due to advances in molecular technology, genomics, and metagenomics, microorganisms of all forms, including bacteria, fungi, helminths, protozoa, and viruses can be detected and identified based upon specific, universal or semi-universal (conserved and semi-conserved) genes or transcripts. Novel methods have been developed and disclosed herein to improve diagnostics and empower the goals of subject-specific treatments into modern day practice.

Level I: A rapid panel or multiplex assay to identify key microbes/pathogens, provide absolute or relative abundance information and generate baseline quantitative measurements of the microorganisms.

Level II: A comprehensive highly parallel and/or multiplexed sequencing approach to determine genetic information associated with a specimen thereby allowing the microbial content of the specimen to be evaluated.

Types of primers: Universal, specific, semi-universal, targeting kingdoms, super-kingdoms, targeting phylums, targeting all classes, orders, families, genera, or species of microorganisms.

Tags: Types of tags are selected oligonucleotides that may be from 2 nucleotides to 200 nucleotides in length (preferably from 6 nucleotides to 12 nucleotides in length) and are used to tag, identify, barcode, or define which sequences are derived from which specimen.

Database formation: a nucleotide or protein database containing genetic information from all known microorganisms, formatted or raw to promote comparison of sequencing data to known or existing data for use in identifying microorganisms, characterizing microbial populations.

An apparatus and method are provided for performing DNA extraction from a specimen, then performing a PCR panel Level I microbial pathogen screening to identify and quantify a specific set or panel of microorganisms or pathogens and genetic antibiotic resistance factors. This is followed by a comprehensive method of microbial tag-encoded FLX (or similar pyrosequencing apparatus) amplicon pyrosequencing that can detect and identify, through computational or bioinformatics methods, the profile of microorganisms within the specimen. The method further, utilizes a database of known sequence information to compare against sequence information derived from the specimen to identify which microorganisms are present in the specimen. This in turn is followed by subsequent computational or bioinformatics algorithms which draws from a database containing information on pathogen-sensitivity to one or more antimicrobial agents or treatments, antibiotic resistance, and previous treatment outcomes to obtain a profile of those antibiotics, antibiofilm agents (e.g., Sanguitec® or LipoGel® gels, lactoferrin, EDTA, gallium salts, xylitol, farnesol, and hamamelitannin), growth factors, cultured cells, probiotics, phages, chemicals, silencing RNAs or DNAs, miRNAs, RNAs, DNAs, vaccines, antibodies, or other therapeutics, which may be utilized to treat or positively impact the microbial profile identified. The computational system then generates interpretive diagnostic and ecology reports that elucidate the microbial composition of the specimen and provide the associated therapeutic options. The apparatus and method comprises a comprehensive microbial diversity identification and evaluation system to guide personalized treatments for infections and to evaluate the microbial diversity of complex patient or environmental systems.

In broad terms, a preferred embodiment of the diagnostic and microbial ecology method is the employment of a rapid (Level I) polymerase chain reaction test utilizing a targeted microbial and genetic resistance factor detection panel most preferably specific to targets identified by molecular surveys for the environment or tissue site of interest, a second preferred embodiment of the method employs an efficient (Level II) comprehensive pyrosequencing diagnostic approach to identify microorganisms not specifically targeted by the Level I panel, followed by a computational system to characterize the microbial and genetic resistance profile and provide reports and interpretations. Each of these diagnostic levels may be utilized independently, but are preferred in combination for comprehensive analysis.

An advantage of some embodiments is that it provides a cost effective molecular diagnostic method and microbial ecology characterization method. This improves the ability of clinicians to treat infections including polymicrobial and biofilm phenotype infections, not conducive to diagnosis by traditional culture-based methodology. Another advantage is the ability to utilize the microbial profiles to determine which antibiotics may be utilized to most efficiently and effectively control or treat an infection in a comprehensive and rapid manner. Another advantage is that computational methods provide a diagnostic and therapeutic interpretive report that can be utilized by a clinician to personalize therapies for each subject or even independent sites on the same subject. Another advantage is that use of this methodology has shown the ability to improve the healing rate of infections. Another advantage is that this method does not rely on the ability of a microorganism to be grown in the laboratory. Another advantage is that hard to culture, fastidious organism, organisms in biofilm phenotype and viable but non-culturable organism can be identified and all organisms can be quantified or relatively quantified. Another advantage is that patient-specific therapeutic regimes can be identified for clinicians to address the complex nature of polymicrobial or poor culturing microbial infections. Another advantage is that an algorithm for identifying such therapeutics, which can best target a specific microbial polymicrobial infection, can be determined.

Disclosed herein are apparati and methods for identifying and determining the amount of two or more pathogens in an individual subject or specimen, including asymptomatic subjects and subjects, who are immunocompromised or immunosuppressed, but asymptomatic with respect to the pathogenic disease(s) of interest, in order to monitor or diagnose or develop information relative to disease emergence and/or disease progression, and to evaluate the microbial diversity and evaluate the microbial ecology of any specimen where there are microorganisms present.

In one aspect, the apparati and methods disclosed herein permit identifying the presence and/or the relative or the specific quantity of two or more microorganisms, particularly bacterial, fungal, helminthal, protozoan or viral pathogens, that may be present in a given environmental or biological specimen. The methods perform such utility through the individual or combined use of quantitative PCR and multiplexed or highly parallelized sequencing or pyrosequencing of directly extracted RNA or DNA from the environmental or biological specimen.

The apparati and methods permit the detection and quantification of pathogens or microorganism via specific polynucleotides, e.g., DNAs or RNAs isolated from an environmental, biological, or clinical specimen, both within a panel of reactions, in a multiplex format and in a highly parallelized sequencing pyrosequencing or future sequencing format, that can further permit the determination of levels (e.g., ratios, percentages, and quantities) for two or more target polynucleotides in a single reaction. Identification and quantification of pathogen specific targets in a specimen has a myriad clinical and microbial ecology utilities specifically to identification of differences between environments, to identify infection-specific or patient-specific therapies.

In one aspect, the apparati and methods described herein use or generate amplification products of known sizes that both differ from each other at the sequence level in specific regions of the polynucleotide and are the same or similar or conserved (same) in specific regions of the polynucleotide. Further, a set of oligonucleotide primers that are specific and target a DNA or RNA molecule isolated from the specimen that can be used to identify a given strain, species, genus, family, order, class, or phylum of microorganism by targeting non-conserved or conserved regions of a gene or part of the genetic material of the organism or a combination of the two.

In one aspect, the apparati and methods described herein relate to methods of estimating or determining the identification and/or quantification of microorganisms in a specimen following isolation (e.g., extraction or purification) of polynucleotides from the specimen, the method comprising: for a given pathogen specific target polynucleotide, selecting a pair of amplification primers that will generate a target amplicon of known length upon amplification of the target, e.g., by PCR or RT-PCR. The method will provide a relative or absolute quantification of the amount of the target, e.g., by quantitative PCR or RT-PCR or other format of polymerase chain reaction.

In one aspect, apparati and methods described herein relate to the detection of selected pathogens in pre-symptomatic immunocompromised or immunosuppressed subjects. Since development of clinical symptoms can be subclinical in many infections and in immunosuppressed subjects, particularly transplant recipients undergoing immunosuppressant therapy, quantitative rapid and or comprehensive detection of bacterial, fungal, helminthal, protozoan, and viral pathogens provides a means to guide therapy during the early stages of infection.

In one aspect, the apparati and methods analyze a specimen suspected of containing any of a polymicrobial community of predetermined or unknown pathogens by screening a specimen for a known and unknown pathogens specific, universal, semi-universal or conserved targets to be used in a nucleic acid amplification reaction to produce an amplicon from each pathogen specific target. The methods include selecting a series of pathogen-specific or kingdom based universal or semi-universal primer pairs wherein each primer pair corresponds to and is targeted to polynucleotide sequences specific to a corresponding pathogen or conserved or universal for all known or unknown microorganisms. The series of pathogen-specific primers or universal or semi-universal domain, kingdom, phylum, class, order, family, genus, or species specific primers when used together produce amplicons of distinct sizes such that the presence of a specific or group of known or unknown pathogen in the specimen. Amplicons are detected by resolving a portion of the amplification mixture to determine if amplicons are present, and is so, their size and then amount of amplicon. Portions of the specimen may be sampled at intermediate points during amplification to determine when amplicons are first detectable, or at the end of amplification. Portions of the specimen may be sampled for downstream sequencing.

In one aspect, the apparati and methods for quantifying a plurality of predetermined pathogens in a specimen suspected of containing at least one pathogen. The methods include obtaining a specimen suspected of containing at least one of the predetermined pathogens. The specimen may be obtained from the environment (e.g., soil, water, animal or human waste), from a plant, animal, frozen tissue banks, or human source (e.g., a pathogen carrier or host). Polynucleotides are isolated from the specimen for use as target in an amplification reaction to produce template. Pathogen-specific or universal or semi-universal primers are selected to correspond to each or all of the plurality of pathogens that could be present in the specimen. Control polynucleotides, preferably competitor polynucleotides, may also be included in the amplification reaction. The competitor polynucleotides can be templates for amplification by pathogen-specific primers, but produce amplicons of a distinct size from the products amplified from the specifically targeted or universal or semi-universal oligonucleotide primers using the same or any other pathogen-specific universal or semi-universal oligonucleotide primers with specimen-derived or control templates. Competitor polynucleotides are added at multiple specific but differing concentrations (i.e., copy numbers) to allow for determination or estimation of the quantity (i.e., copy number) of a pathogen-specific, universal or semi-universal nucleic acid amplifications generated from the specimen.

In one aspect, the apparati and methods include monitoring of a series of specimens from the same source for any of a predetermined plurality or multiplicity of pathogens. The methods include obtaining a specimen from a source at regular intervals (e.g., about continually, hourly, daily, weekly, about monthly, about quarterly or yearly) and quantifying the amount or relative amount of the composition of pathogen or multiple pathogens or specific or unknown organisms in the specimen using any amplification method and also followed by sequencing or pyrosequencing approaches utilizing tagging methodologies, including bTEFAP methods. Sequencing of greater than 50 nucleotides is preferred, greater than 250 nucleotides is preferred, and greater than 400 nucleotides is even more preferred. A source may be any specimen suspected clinically of containing microorganisms. By evaluating the microbial composition and relative or absolute abundance of pathogens at discrete, random or regular intervals, pathogens may be detected in the asymptomatic individual and appropriate measures can be taken, such as modification of administration of compositions that result in immunosuppression of the individual or administration of a therapy to ameliorate and/or treat the pathogen infection.

### DEFINITIONS

The term “prepared or isolated from” when used in reference to polynucleotides “prepared or isolated from” a pathogen refers to both polynucleotides (e.g., DNA or RNA, including cDNA produced therefrom) extracted and/or purified from a microorganism, and to polynucleotides that are copied from the transcriptosome of a microorganism, e.g., by a process of reverse-transcription or DNA polymerization using native DNA or RNA as a template. Polynucleotides of the pathogen may be isolated from a specimen in conjunction with host nucleic acid.

“Pathogen” refers to a microorganism, which causes disease in another organism (e.g., animal or plant) by directly infecting the other organism, or by producing agents that causes disease in another organism (e.g., bacteria that produce pathogenic toxins and the like). As used herein, pathogens include, but are not limited to bacteria, fungi (e.g., molds and yeasts), helminths (e.g., cestodes, nematodes, and trematodes), protozoa, viroids and viruses, or any combination thereof, wherein each pathogen is capable, either by itself or in concert with another pathogen, of eliciting disease in vertebrates including but not limited to mammals, and including but not limited to humans. As used herein, the term “pathogen” also encompasses microorganisms, which may not ordinarily be pathogenic in a non-immunocompromised or immunosuppressed host. Specific nonlimiting examples of bacterial pathogens include the species listed in the microbial surveys of the examples. Specific nonlimiting examples of viral pathogens include herpes simplex virus (HSV) 1, HSV2, Epstein Barr virus (EBV), cytomegalovirus (CMV), human herpes virus (HHV) 6, HHV7, HHV8, varicella zoster virus (VZV), hepatitis C, hepatitis B, adenovirus, Eastern Equine Encephalitis Virus (EEEV), West Nile virus (WNE), JC virus (JCV), and BK virus (BKV), as well as the species listed in the microbial surveys included in this disclosure. “Microorganism” includes prokaryotic and eukaryotic microbial species from the Domains of Archaea, Bacteria, and Eucarya, the latter including yeast and filamentous fungi, helminths, protozoa, algae, or higher Protista. The term “microbe” is used interchangeably with the term microorganism.

“Bacteria” or “Eubacteria” refers to a domain of prokaryotic organisms. Bacteria include at least 11 distinct groups as follows: (1) Gram-positive (gram+) bacteria, of which there are two major subdivisions: (i) high G+C group (Actinomycetes, Mycobacteria, Micrococcus, others) (ii) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram-positive bacteria” include cocci, nonsporulating rods, and sporulating rods. The genera of Gram-positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

“Detection” refers to the at least qualitative determination of the presence or absence of a microorganism in a specimen. The term “identification” also includes the detection of a microorganism, i.e., determining the genus, species, or strain of a microorganism according to its recognized taxonomy in the art and as described in the present specification. The term “identification” further includes the quantification of a microorganism in a specimen, e.g., the copy number of the microorganism in a microliter (or a milliliter or a liter) or a microgram (or a milligram or a gram or a kilogram) of a specimen.

The term “analyzing” when used in the context of an amplification reaction refers to a qualitative (i.e., presence or absence, size detection, or identity etc.) or quantitative (i.e., amount) determination of a target polynucleotide, which may be visual or automated assessments based upon the magnitude (strength) or number of signals generated by the label. The “amount” (e.g., measured in μg, μmol, or copy number) of a polynucleotide may be measured by methods well known in the art (e.g., by UV absorption or fluorescence intensity, by comparing band intensity on a gel with a reference of known length and amount), for example, as described in Basic Methods in Molecular Biology (1986, Davis et al., Elsevier) and Current Protocols in Molecular Biology (1997, Ausubel et al., John Wiley). One way of measuring the amount of a polynucleotide in one embodiment is to measure the fluorescence intensity emitted by such polynucleotide, and compare it with the fluorescence intensity emitted by a reference polynucleotide, i.e., a polynucleotide with a known amount.

“Plurality” refers to two or more, for example, at least three, at least four, at least five, at least six, at least seven, at least eight, at least nine, at least ten, etc.

“Specimen” refers to a biological material, which is isolated from its natural environment (including the body such as skin, ear, nose, sinus, throat, mucosa of the respiratory or urogenital system, internal organs such as the cardiovascular or gastrointestinal system, bone, feces, and fluids or a body cavity collected by lavage) and contains a plurality of polynucleotides. For example, the specimen may be genetic material obtained from exudate of a wound or cutaneous infection, removed from the wound or cutaneous infection, or biopsy or surgically excised tissue. A biological fluid includes, but is not limited to, blood, plasma, serum, sputum, urine, abscess, pus or other wound exudate, infected tissue sampled by wound debridement or excision, cerebrospinal fluid, lavage, and leucopoiesis specimens, for example. A specimen may also be an environmental specimen such as soil, water, or animal or human waste to detect the presence of a pathogen in an area where an outbreak of disease related to a specific pathogen has occurred. A specimen may also be obtained from a tissue bank or other source for the analysis of archival samples or to test samples prior to transplantation. A specimen useful in the methods described herein may be any plant, animal, bacterial, fungal, helminthal, protozoan, or viral material containing a plurality of polynucleotides, or any amplified templates, genomes, or metagenomes derived therefrom.

A specimen is suspected of containing at least one of a plurality of known or unknown or potential or opportunistic pathogens or commensal organisms for any of a number of reasons. For example, a soil specimen may be suspected of containing a pathogen if humans or animals living close to the location where the soil specimen was collected show symptoms of a condition or diseases associated with a soil pathogen. Few environments and therefore few specimens are sterile and do not contain some type of microorganism. Thus, a specimen is any collection of source material sampled from any environment. Specimens taken from such a subject may be suspected of containing at least one of a plurality of known unknown, suspected, opportunistic or potential pathogens or commensal organisms, even in the absence of infection. A subject who is “immunocompromised” or “immunosuppressed” refers to a subject who is at risk for developing infectious diseases, because of an immune deficiency. The subject may be immunosuppressed due to a treatment regimen designed, for example, to prevent inflammation or to prevent rejection of a transplant.

The term “asymptomatic” refers to a subject who does not exhibit physical symptoms characteristic of being infected with a given pathogen, or a given combinations of pathogens.

A primer pair “capable of mediating amplification” is understood as a primer pair that is specific to a target polynucleotide, has an appropriate melting temperature, and does not include excessive secondary structure. Guidelines for designing primer pairs capable of mediating amplification are well documented in the literature. There are also linear amplification methods and sequencing that is usually performed in cycles using a single primer.

“Conditions that promote amplification” are the conditions for target amplification provided by the manufacturer for the enzyme used for amplification of template. It is understood that an enzyme may work under a range of conditions (e.g., buffer pH, ion concentrations, temperatures, concentrations of enzyme or target). It is also understood that several temperatures may be required for amplification (e.g., three in PCR for annealing primer to template, extending primer as the complement of template, and denaturing extended primer from template). Conditions that promote amplification need not be identical for all primers and targets in a reaction, and reactions may be carried out under suboptimal conditions where amplification is still possible.

“Separating” nucleic acids in a sample refers to a process whereby they are separated by size (i.e., length). The method of separation should be capable of resolving nucleic acid fragments that differ in size by ten nucleotides or less (or, alternatively, by ten base pairs or less, e.g., where non-denaturing conditions are employed). Preferred resolution for separation techniques employed in the methods described herein includes resolution of nucleic acids differing by five nucleotides or less (alternatively, five base pairs or less), up to and including resolution of nucleic acids differing by only one nucleotide (or one base pair).

“Amplified product” refers to polynucleotides that are entire or partial copies of a target polynucleotide, produced in an amplification reaction. An “amplified product” according to the one embodiment, may be DNA or RNA, and it may be double-stranded or single-stranded. An amplified product is also referred to herein as an “amplicon.”

“Amplification” or “amplification reaction” refers to a reaction for generating a copy of a particular polynucleotide sequence or increasing the copy number or amount of a particular polynucleotide sequence. For example, polynucleotide amplification may be a process using a polymerase and a pair of oligonucleotide primers for producing any particular polynucleotide sequence, i.e., the whole or a portion of a target polynucleotide sequence, in an amount that is greater than that initially present. Amplification may be accomplished by the in vitro methods of the polymerase chain reaction (PCR). See generally, PCR Technology: Principles and Applications for DNA Amplification (Erlich, ed.) Freeman (1992); PCR Protocols: A Guide to Methods and Applications (Innis et al., eds.) Academic (1990); Mattila et al., 1991, Nucleic Acids Res. 19: 4967; Eckert et al., 1991, PCR Methods and Applications 1: 17; PCR (McPherson et al., eds.), IRL Press (1995); and U.S. Pat. Nos. 4,683,202 and 4,683,195, each of which is incorporated by reference in its entirety. Other amplification methods include, but are not limited to: (a) ligase chain reaction (LCR) (see Wu & Wallace, 1989, Genomics 4: 560-569; Landegren et al., 1988, Science, 241: 1077-1080); (b) transcription amplification (Kwoh et al., 1989, Proc. Natl. Acad. Sci. USA 86: 1173-1177); (c) self-sustained sequence replication (Guatelli et al., 1990, Proc. Natl. Acad. Sci. USA, 87: 1874-1878); and (d) nucleic acid based sequence amplification (NABSA) (Sooknanan & Malek, 1995, Bio/Technology 13: 563-565), each of which is incorporated by reference in its entirety.

A “target polynucleotide” (including, e.g., a target RNA, target cDNA, or target DNA) is a polynucleotide to be analyzed. A target polynucleotide may be isolated or amplified before being analyzed. For example, the target polynucleotide may be comprised of a sequence that lies between the hybridization regions of two members of a pair of oligonucleotide primers that are used to amplify the target. A target polynucleotide may be RNA or DNA (including, e.g., cDNA).

A “microbe-specific target polynucleotide” is a target polynucleotide as defined above, wherein the target polynucleotide is prepared or isolated from a specimen suspected of containing a pathogen, and which is present in only one member of the group of different pathogens that are being analyzed (i.e., the target polynucleotide has a unique sequence and is specific for detection of the pathogen's genera or species).

An “oligonucleotide primer” refers to a polynucleotide molecule (i.e., DNA or RNA) capable of annealing to a polynucleotide template and providing a 3′-end to produce an extension product that is complementary to the polynucleotide template. The conditions for initiation and extension usually include the presence of four different deoxyribonucleoside triphosphates (dNTPs) and a polymerization-inducing agent such as a DNA polymerase or reverse transcriptase activity, in a suitable buffer (“buffer” includes substituents which are cofactors, or which affect pH, ionic strength, etc.) and at a suitable temperature. The primer as described herein may be single- or double-stranded. The primer is preferably single-stranded for maximum efficiency in amplification. “Primers” useful in the methods described herein are less than or equal to 100 nucleotides in length, e.g., less than or equal to 90, or 80, or 70, or 60, or 50, or 40, or 30, or 20, or 15, but preferably longer than 10 nucleotides in length.

“Label” or “detectable label” refers to any moiety or molecule that can be used to provide a detectable (preferably quantifiable) signal. A “labeled nucleotide” (e.g., a dNTP) or “labeled polynucleotide” is one linked to a detectable label. The term “linked” encompasses covalently and non-covalently bonded, e.g., by hydrogen, ionic, or Van der Waals bonds. Such bonds may be formed between at least two of the same or different atoms or ions as a result of redistribution of electron densities of those atoms or ions. Labels may provide signals detectable by fluorescence, radioactivity, colorimetry, gravimetry, X-ray diffraction or absorption, magnetism, enzymatic activity, mass spectrometry, binding affinity, hybridization radiofrequency, nanocrystals, and the like. A nucleotide useful in the methods described herein can be labeled so that the amplified product may incorporate the labeled nucleotide and becomes detectable. A fluorescent dye is a preferred label according to the one embodiment. Suitable fluorescent dyes include fluorochromes such as Cy5, Cy3, rhodamine and derivatives (such as Texas Red), fluorescein and derivatives (such as 5-bromomethyl fluorescein), Lucifer Yellow, IAEDANS, 7-Me.sub.2N-coumarin-4-acetate, 7-OH-4-CH.sub.3-coumarin-3-acetate, 7-NH₂-4-CH₃-coumarin-3-acetate (AMCA), monobromobimane, pyrene trisulfonates, such as Cascade Blue, and monobromorimethyl-ammoniobimane (see, for example, DeLuca, 1982, Immunofluorescence Analysis, in Antibody As a Tool, Marchalonis, et al., eds., Wiley, which is incorporated herein by reference).

It is intended that “labeled nucleotide” as used herein also encompasses a synthetic or biochemically derived nucleotide analog that is intrinsically fluorescent, e.g., as described in U.S. Pat. Nos. 6,268,132 and 5,763,167, Hawkins et al. (1995, Nucleic Acids Res., 23: 2872-2880), Seela et al. (2000, Helvetica Chimica Acta, 83: 910-927), Wierzchowski et al. (1996, Biochimica et Biophysica Acta, 1290: 9-17), Virta et al. (2003, Nucleosides, Nucleotides & Nucleic Acids, 22: 85-98), the entirety of each is hereby incorporated by reference. By “intrinsically fluorescent” it is meant that the nucleotide analog is spectrally unique and distinct from the commonly occurring conventional nucleosides in their capacities for selective excitation and emission under physiological conditions. For the intrinsically fluorescent nucleotides, the fluorescence typically occurs at wavelengths in the near ultraviolet through the visible wavelengths. Preferably, fluorescence will occur at wavelengths between 250 nm and 700 nm and most preferably in the visible wavelengths between 250 nm and 500 nm.

The “detectable label” or “label” includes a molecule or moiety capable of generating a detectable signal (i.e. light), either by itself or through the interaction with another label. The “label” may be a member of a signal generating system, and thus can generate a detectable signal in context with other members of the signal generating system, e.g., a biotin-avidin signal generation system, or a donor-acceptor pair for fluorescent resonance energy transfer (FRET) (Stryer et al., 1978, Ann. Rev. Biochem., 47: 819-846; Selvin, 1995, Methods Enzymol., 246: 300-334) or a nucleic acid-binding dye, producing detectable signal upon binding to polynucleotide (DNA or RNA molecule).

A “nucleotide” refers to a phosphate ester of a nucleoside, e.g., mono-, di, -tri-, and tetraphosphate esters, wherein the most common site of esterification is the hydroxyl group attached to the C-5 position of the pentose (or equivalent position of a non-pentose “sugar moiety”). The term “nucleotide” includes both a conventional nucleotide and a non-conventional nucleotide which includes, but is not limited to, phosphorothioate, phosphite, ring atom modified derivatives, and the like, e.g., an intrinsically fluorescent nucleotide. The term “conventional nucleotide” refers to one of the “naturally occurring” deoxynucleotides (dNTPs), including dATP, dTTP, dCTP, dGTP, dUTP, and dITP whereas the term “non-conventional nucleotide” refers to a nucleotide, which is not a naturally occurring nucleotide. The term “naturally occurring” refers to a nucleotide that exists in nature without human intervention. In contradistinction, the term “non-conventional nucleotide” refers to a nucleotide that exists only with human intervention. A “non-conventional nucleotide” may include a nucleotide in which the pentose sugar and/or one or more of the phosphate esters is replaced with a respective analog. Nonlimiting examples of pentose sugar analogs are those previously described in conjunction with nucleoside analogs. Nonlimiting examples of phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., including any associated counterions, if present. A non-conventional nucleotide may show a preference of base pairing with another artificial nucleotide over a conventional nucleotide (see Ohtsuki et al., 2001, Proc. Natl. Acad. Sci., 98: 4922-4925). The base pairing ability may be measured by the T7 transcription assay as described in Ohtsuki et al. (2001). Other non-limiting examples of “artificial nucleotides” may be found in Lutz et al. (1998, Bioorg. Med. Chem. Lett., 8: 1149-1152); Voegel & Benner (1996, Helv. Chim. Acta 76: 1863-1880); Horlacher et al. (1995, Proc. Natl. Acad. Sci., 92: 6329-6333); Switzer et al. (1993, Biochemistry 32: 10489-10496); Tor & Dervan (1993, J. Am. Chem. Soc. 115: 4461-4467); Piccirilli et al. (1991, Biochemistry 30: 10350-10356); Switzer et al. (1989, J. Am. Chem. Soc. 111: 8322-8323), all of which are hereby incorporated by reference. A “non-conventional nucleotide” may also be a degenerate nucleotide or an intrinsically fluorescent nucleotide.

“Degenerate nucleotide” means a nucleotide that may be able to basepair with at least two bases of dA, dG, dC, and dT. A non-limiting list of degenerate nucleotides that basepairs with at least two bases of dA, dG, dC, and dT include: inosine, 5-nitropyrole, 5-nitroindole, hypoxanthine, 6H,8H,4-dihydropyrimido[4,5c][1,2]oxacin-7-one (P), 2-amino-6-methoxyaminopurine, dPTP, and 8-oxo-dGTP.

“Opposite orientation” refers to one nucleotide sequence complementary to the sense strand of a target polynucleotide template and another nucleotide sequence complementary to the antisense strand of the same target polynucleotide template. Primers with opposite orientation may generate a PCR-amplified product from matched polynucleotide template to which they complement. Two primers having opposite orientation may be referred to as a “reverse” primer and a “forward” primer.

“Same orientation” means that primers comprise nucleotide sequences complementary to the same strand of a target polynucleotide template. Primers with same orientation will not generate a PCR-amplified product from matched polynucleotide template to which they complement.

“Polynucleotide” or “nucleic acid” refers to a polymerized deoxyribonucleotide or ribonucleotide, which may be unmodified RNA or DNA or modified RNA or DNA. They include without limitation single- and double-stranded polynucleotides, and embrace chemically, enzymatically, or metabolically modified forms of polynucleotides, as well as chemical forms of DNA and RNA characteristic of particles and cells. A polynucleotide may be an isolated or purified polynucleotide or it may be an amplified polynucleotide in an amplification reaction.

“Set” refers to a group of at least two. Thus, a “set” of oligonucleotide primers comprises at least two oligonucleotide primers. In one aspect, a “set” of oligonucleotide primers refers to a group of primers sufficient to specifically amplify a nucleic acid amplicon from each member of a plurality of target pathogens—generally, there will be a pair of oligonucleotide primers for each member of said plurality, (it is noted that these primer pairs will, in some aspects, also be used to amplify one or more competitor or internal standard templates).

“Pair” refers to two. Thus, a “pair” of oligonucleotide primers are two oligonucleotide primers. When a “pair” of oligonucleotide primers are used to produce an extended product from a double-stranded template (e.g., genomic DNA or cDNA), it is preferred that the pair of oligonucleotide primers hybridize to different stand of the double-stranded template, i.e., they have opposite orientations.

“Isolated” or “purified” means that a naturally-occurring substance was removed from its normal cellular environment or is synthesized in a non-natural environment (e.g., artificially synthesized). Thus, an “isolated” or “purified” substance may be in a cell-free solution or placed in a different cellular environment. For example, “purified” does not necessarily imply that a sequence is the only nucleotide present, but that it is essentially free (at least about 90% or 95%, up to 99-100% pure) of non-nucleotide or polynucleotide material naturally associated with it.

“cDNA” refers to complementary or copy polynucleotide produced from an RNA template by the action of an RNA-dependent DNA polymerase activity (e.g., reverse transcriptase).

“Complementary” refers to the ability of a single strand of a polynucleotide (or portion thereof) to hybridize to an anti-parallel polynucleotide strand (or portion thereof) by contiguous base-pairing between the nucleotides (that is not interrupted by any unpaired nucleotides) of the anti-parallel polynucleotide single strands, thereby forming a double-stranded polynucleotide between the complementary strands. A first polynucleotide is said to be “completely complementary” to a second polynucleotide strand if each and every nucleotide of the first polynucleotide forms base-paring with nucleotides within the complementary region of the second polynucleotide. A first polynucleotide is not completely complementary (i.e., partially complementary) to the second polynucleotide if one nucleotide in the first polynucleotide does not base pair with the corresponding nucleotide in the second polynucleotide. The degree of complementarity between polynucleotide strands has significant effects on the efficiency and strength of annealing or hybridization between polynucleotide strands. This is of particular importance in amplification reactions, which depend upon binding between polynucleotide strands.

An oligonucleotide primer is “complementary” to a target polynucleotide if at least 50% (preferably, 60%, more preferably 70%, 80%, still more preferably 90% or more) nucleotides of the primer form base pairs with nucleotides on the target polynucleotide.

The apparati and methods described here utilize both a rapid Level I quantitative PCR panel containing a specific set or sets of oligonucleotides, preferably identified by molecular microbial survey, to diagnose and quantify specific individual pathogens in a multiplex or highly parallelized format, incorporated with simultaneous universal probe sets that allow for quantification of total numbers of pathogens, commensals, opportunistic pathogens, potential pathogens, unknown pathogens or suspected pathogens. Together the specifically targeted quantitative PCR assays are multiplexed together in panels along with the universal kingdom level assays thereby providing both a relative quantification of each specific pathogen but also estimates of relative abundance and quantification of the total microorganism load in a sample.

The apparati and methods described here utilize a comprehensive Level II assay that can identify, provide relative quantification, relative abundance or absolute identification/resolution of these quantitative factors of all known, unknown, suspected, commensal, opportunistic, pathogens and microorganisms using a bTEFAP technique.

The apparati and methods described herein include both the Level I and Level II molecular assays that can work together or independently. These assays provide diagnostic, monitoring, evaluation and screening using oligonucleotide probes and primers to amplify organism-specific, universal, or semi-universal portions of the genes or genomes of selected, specific or all pathogens (pathogens may be suspected pathogens, unknown or previously undescribed, unrecognized, or unappreciated pathogens, opportunistic pathogens, commensal organisms that provide synergistic contribution to pathogenicity and polymicrobial communities that act together to create infection or subclinical disease including organisms in biofilm or any other phenotype or compilation within a sample hereafter referred to as pathogens) contained within a sample. The pathogen is selected from the group consisting of: bacteria, fungi (e.g., molds and yeasts), helminths, protozoan, viruses, and combinations thereof. Preferably, the pathogen is selected from the group consisting of: bacteria, fungi, viruses, and combinations thereof. Alternatively, the pathogen is selected from the group consisting of: bacteria, viruses, and combinations thereof. More preferably, the pathogens may be microbes belonging to at least two, at least three, at least four, at least five, at least six, at least seven, at least eight, at least nine, or at least ten different genera (especially bacterial and/or viral genera); the pathogens may be bacteria belonging to at least five, at least ten, at least 15, at least 20, at least 25, at least 30, at least 35, at least 40, at least 45, or at least 50 different species (especially bacterial and/or viral species).

The apparati and methods describe methods for evaluating an infection. An infection may be a suspected infection, subclinical infection, a potential infection, a future infection, or a past infection hereafter referred to as infection. A specimen may be from any environment including bodily fluids, feces, tissue, debrided materials, swabbed surfaces, biopsies, aqueous materials, fluids collected from any source, surfaces of any type, soil, food, etc., including any environment that contains microorganisms. A specimen is any form of content removed in whole or in part from an environment intended for analysis of microorganisms using Level I and/or Level II molecular assays.

Diagnostic, screening, monitoring, or testing for pathogens causing an infection is typically conducted for a subject who presents symptoms characteristic of clinical infection presumably by one or more pathogenic microorganisms, or in a subject who has been in contact with another having one or more pathogenic infections, or in a subject who is otherwise suspected to have developed an infectious disease resulting from one or more pathogens.

Many pathogens causing an infection or present in a specimen may be unknown. The literature suggests that only 5%-10% of microorganisms have been characterized and fully identified. Level I may be utilized to target these known pathogens or pathogen panels identified by molecular survey as prevalent in a particular environment utilizing a multiplex, parallel or panel format allowing such pathogens to be detected and quantified rapidly. The unknown organisms can be detected, and their relationship to known organisms defined, allowing a previously unrealized ability to define infections caused by unknown microorganisms. Further, the Level II assay is also able to define, detect and identify known, suspected and other types of pathogens.

While quantitative monitoring of pathogens in asymptomatic individuals is not generally practical (especially using traditional methods), it can be very beneficial for subjects undergoing immunosuppressive treatment considering the accuracy and efficiency of the methodology disclosed herein. Quantitative pathogen monitoring in a subject is especially practical, if applied not as a single test for each specific infection of interest, but if applied as a panel of parallel assays using Level I testing and/or as a comprehensive universal diagnostic such as Level II testing. Such diagnostics or monitoring can be performed on a single specimen from a subject and, preferably, as a multiplex assay for a panel of pathogens. In the case of only targeting known pathogens, assays do not represent novel panels, but combined with the benefits of a comprehensive universal diagnostic this represents a never before described format, method and technology.

Hence, the Level I and Level II assays were developed using molecular diagnostics methods, and, in particular, methods using PCR amplification of pathogen-specific polynucleotides and their high-throughput sequencing. After extraction of nucleic acids, all analytical steps may be completed in less than 72 hours. Where the diagnostic results (e.g., a genus, species, or strain of microorganism was or was not detected) are tabulated into a report in less than 96 hours; the report may listing a genus, species, or strain of microorganism as detected or below the sensitivity of detection. The sensitivity may be more than 25, more than 50, more than 75, more than 100, more than 150, more than 250, or more than 500 of the target polynucleotide. The specimen may be analyzed in less than 12 hours, less than 24 hours, less than 36 hours, less than 48 hours, less than 60 hours, less than 72 hours, less than 84 hours, or less than 96 hours from collection. The report may be sent to the treating physician in less than 12 hours, less than 24 hours, less than 36 hours, less than 48 hours, less than 60 hours, less than 72 hours, less than 84 hours, or less than 96 hours from collection.

### EXAMPLES

### Example 1

### General

### Tag Design

Tags were designed to have a polynucleotide sequence that is unique to each specimen.

Either manual design or computer-aided design may be utilized. Tags may also be referred to as barcodes, alien fragments, fragments etc. Their purpose is to artificially and uniquely label molecular reactions performed on a specimen to enable specific evaluation of the specimen by downstream computational or bioinformatics algorithms. A tag may be from 2 nucleotides to 1000 nucleotides with a preferred length from 6 nucleotides to 12 nucleotides. As an example, PCR performed on Sample A may be tagged with the 8 nucleotide tag ACCGTCAT (SEQ ID NO:1). This tag subsequently identifies Sample A within the downstream processing. Sample B is tagged with the 6 nucleotide tag AGCGTC (SEQ ID NO:2). For N samples to be analyzed in parallel or in a multiplex process, N tags equal to the total number of samples are used. Thus, based upon these unique tags, samples can be distinguished even if they have similar microbial populations.

### Oligonucleotide Design and Synthesis

Primers were designed to target specific or groups of microorganisms. As an example, Sample A and Sample B may be evaluated for all or most members of the kingdom eubacteria (bacteria), archaebacteria (archaea), and the group of eukaryotes known as fungi (metazoa). In these examples, a region of bacterial genomes, which is conserved among all bacteria (e.g., 16S ribosomal DNA, SSU ribosomal RNA, large subunit ribosomal DNA or RNA, DNA repair gene, etc.), will be aligned and utilize two conserved regions for this gene located on either side of a variable genetic region primers designed to target the conserved region. For example, it is understood by those skilled in the art that the SSU or 16S ribosomal subunit of bacteria (or 18S ribosomal subunit of yeast) has a number of highly conserved regions (considered to have a polynucleotide composition largely similar among all microorganisms in a kingdom or other taxonomic designation) and a number of genetically variable regions (considered to have composition largely unique to individual species, genus, or other taxonomic designation). Primers designed for two or more conserved regions, or more degenerate primers designed to portions of one or more variable regions, may be utilized to amplify the ribosome sequence of all or specific groups (or specific taxonomic level) of bacteria within a specimen. Similarly, with groups of microorganisms such a metazoan, algae, archaea, protozoa, and viruses such an approach may be utilized to design universal, semi-universal or taxonomic level specific primers. In a preferred embodiment, such primers will amplify from two or more conserved regions across one or more variable region. By further sequencing this amplified product, the microbial population present in a specimen may be defined by computation and or bioinformatics analyses of all the sequences. Subsequently, microorganisms present in the specimen are identified and/or quantified based upon their unique sequences. A species- or genus-specific primer pair is designed to target only one species or genera of known microorganisms. In this case, a genetic sequence that is unique to that specific organism is designed such that when amplifying a specimen with a multiplicity of microorganism (two or more different microorganisms) only the organism of interest is amplified. Thus specific detection of an amplicon may be used to indicate that this organism was present and may also be used in conjunction with real-time PCR to provide quantitative information on this specific organism. Similarly, a universal set of primers as described and exemplified above may be used to quantify the consortium (two or more microorganism) present in a specimen.

PCR using linker tags and primers may be performed as one step reactions, two step reactions or multiple step reactions including PCR followed by ligation steps to incorporate a tag or sequencing linkers ultimately generating a sequencing library capable of multiplex and highly parallelized sequencing that encodes specimen-specific tags capable of being utilized in downstream steps for individual sample evaluations of a specific microbial population or all populations or portions of populations (e.g., analyzing only eubacteria (bacteria), or only phylum clostridia or both bacteria and fungi (metazoan), or only fungi (molds and yeasts), or specific taxonomic groups of fungi, or all microorganisms, or groups of pathogens, or a single class or group of eubacteria or archaea or a specific species or strain of bacteria, fungi, helminths, protozoa, viruses, or combinations thereof).

Primers may be selected or designed using software known to those skilled in the art such as PrimerSelect software (DNASTAR), or Oligodesign (Integrated DNA Technologies) based on criteria as provided in the following example: from 12 to 50 nucleotides in length; Melting temperature (Tm) 50.5° C.-60.2° C.; primer stability −50 to −35 kcal per mole; unique primer 3′ sequence of eight nucleotides; avoiding self-primer and primer pair formation longer than two contiguous bases (ignoring duplexing eight bases from 3′-end); avoiding internal primer hairpins longer than two or more bases; with minimal 3′ pentamer stability of −8.0 kcal per mole or more.

In addition, selected primer pairs were assessed for dimer formation in multiplex across different pairs to eliminate any potential dimers with stability less than −7.0 kcal per mole. Furthermore, primers were screened against none-redundant DNA database (Gene Bank, NCBI) using BLAST search program to eliminate any primers with significant (e.g., greater than ten contiguous nucleotides over or five contiguous nucleotides from 3′-end) homology to non-target polynucleotides.

### RT-PCR or PCR or Quantitative PCR Embodiments for Level I

A panel of two or more pathogen specific PCR assays and universal or semi-universal assays for taxonomic groups of microorganisms or kingdoms of microorganisms was performed on polynucleotides extracted from a specimen. Further, a panel of two or more genetic antibiotic resistance factors and a panel of two or more inflammatory markers were performed. These assays, together or in part comprise the Level I assay, which may be utilized independently or combined with the Level II assay for additional utility. RT-PCR may use a chemiluminescent or probe-based method or a sybr green or other like method to detect and provide quantification information on each of the specific microorganisms targeted by individual or multiplex assays in the panel. Similarly the universal or semi-universal primers provide quantitative information.

The Level I assay was run in a PCR panel consisting of multiple individual reactions or wells, conformed in a plate, slide, disk, cartridge or other platform such as Roche 480, Fluidigm Biomark, Qiagen or Biorad PCR or real-time (RT) PCR systems or in a 96, 384, 1536, etc. well, spacing, other compartmentalized, or emulsion based format. Each individual reaction may be multiplexed (having more than one individual pathogen, genetic resistance factor, or inflammatory markers) or having only one such target. A preferred PCR embodiment is a quantitative or relative quantitative set of multiplex of single target assays that are performed on an individual sample, with a single or more than one individual reactions also containing a universal or semi universal amplification target. Together this panel may identify and or provide relative or absolute quantification of specific pathogens, genetic antibiotic resistance factors, and/or inflammatory markers as well as utilizing the universal or semi-universal markers to provide total population quantitative information. Together the specific and universal markers are utilized to provide information on the total population present and to evaluate quantitative or relative quantitative information for the specific or general pathogen, antibiotic resistance factor or inflammatory markers present within a specimen.

Level I assays are processed using relative peaks areas corresponding to target microorganism-specific amplicons and universal or semi-universal targets. Such peaks are plotted as a logarithmic function of PCR cycle number using computational or bioinformatics processes described herein. The linear portion of the each curve (defined as the part of the curve which shows a log linear increase in signal threshold, is extrapolated to an arbitrary threshold (e.g., 1000 relative fluorescent units) to calculate Threshold Cycle (Ct) number. Ct values for known copy numbers of DNA or RNA within the panel that are run as internal control reactions on the same apparatus in parallel or as part of a separate reaction or collected as a database or archive are used to generate a calibration curve and assign relative or absolute quantitative rankings or numbers to each individual or specific target reaction, or universal or semi-universal targets. This data is then utilized to generate a diagnostic report using computational or bioinformatics algorithms or processes. This diagnostic report contains the identity of those targets that were detected, the quantification of the targets, as well as those targets that were analyzed but not detected. This report is considered a Level I diagnostic report (as an example) and may be transmitted along with specific diagnostic and patient information. Also contained within this report is the total abundance of all or specific groups, classes, phylum, kingdoms etc. of microorganisms. Together the specific targets and the universal or semi-universal target results may be used to designate the presence or absence of specific pathogens, genetic antibiotic resistance factors, or inflammatory markers as well as the total abundance of all pathogens, or specific kingdoms, or taxonomomic groups of pathogens.

### Pyrosequencing Embodiments for Level II

The Level II assay is composed of a PCR reaction derived from or separate from level I assays, a set of PCR reactions, or a combination of PCR reactions and molecular ligation reactions that lead to sequencing or pyrosequencing using a parallel or multiplex or massively paralleled technology. Sequencing of greater than 50 nucleotides is preferred, greater than 250 nucleotides is preferred, and greater than 400 nucleotides is even more preferred. Examples of equipment that may be employed include the Roche 454 FLX or subsequent equipment, Helicos technology, GS Apparatus, Illumina HiScanSQ, Illumina Genome sequencer and Pacific Bioscience's SMRT technology, future embodiments of the same or future technologies providing massively parallel sequencing capabilities. The technology as utilized herein was employed to identify, based upon genetic factors, the identity and relative abundance of microorganisms within a specimen. This process begins by utilizing universal, semi-universal, or taxonomic group specific or general primers as described above, that can amplify, as an example, all the pathogens present in a specimen using either specific conserved genes or entire genomes. The PCR is novel in that it incorporates the sequencing linkers (e.g., specific sequences needed to prime a sequencing reaction) a specimen-specific tag, and the universal pathogen, semi-universal pathogen, or taxonomic group primers specific. By way of example, Sample A is screened for all major bacterial and fungal pathogens. In this instance, two or more separate reactions or one multiplex reaction are performed on Sample A using one or more bacterial universal or semi-universal primer set and one or more fungal universal or semi-universal primer set. The final products of these reactions are amplicons, or DNA fragments, that contain the form such as LINKERA-Sample A tag-Forward primer-Unknown pathogen information-Reverse primer-LINKERB (as an example). In this example, the Sample A tag is utilized to specifically mark all sequences that originated from Sample A in any downstream computational or bioinformatics analyses such as identification of pathogens, determining relative abundance of pathogens and predicting or determining antibiotic resistance profiles for pathogens in a specimen. This method is utilized to identity two or more pathogens in a single specimen. When used independently or in combination with the Level I assay this method provides more accurate quantification and characterization of all pathogens present in an infection. Further, the method provides a comprehensive application of the molecular diagnostic methods, including computational methods for analyses which ultimately identify subject- or specimen-specific treatments targeted at the DNA level for each subject's infection and the microbial ecology of the infection.

Sequences generated by the Level II assay are processed using computational or bioinformatics algorithms, which may be encoded in a variety of programming languages and development environments. As an example, custom scripts software or software written in the C# within the Microsoft®.NET (Microsoft Corp, Seattle, Wash.), python, java, C++, among others including programming languages derived within commercial or custom development environment was utilized to generate all possible combinations of 10-mer oligonucleotide tags with GC % between 40 and 60% to provide tags. From this pool, 12 individual tags were selected to label 12 different samples. Custom software developed within the Microsoft®.NET (Microsoft Corp, Seattle, Wash.) environment is utilized for all post sequencing processing. The software takes in sequence quality trimmed sequences (e.g., Phred20 quality or Q20) obtained from the sequencing or pyrosequencing run, which are further processed using a scripted or software based bioinformatics pipeline. Quality trimmed sequencing reads were subsequently derived directly from FLX sequencing run output files. Tags were extracted from the FLX generated multi-FASTA file into individual specimen-specific files based upon the tag sequence. Tags, which did not have 100% homology to the specific designation, were not considered. Sequences, which were less than 120 by after quality trimming, were not considered. The resultant individual specimen FASTA files may then be analyzed as an individual sequence or assembled using an assembly algorithm such as CAP3 (Huang & Madan, 1999, Genome Res. 9: 868-877), NGEN, Seqman, or CLCbio assembler. The ace files or other output format generated by the assembly algorithm, or the individual or representative or consensus sequence, are then processed to generate labeled specimen-specific FASTA file or files containing the tentative consensus (TC) sequences of the assembly or individual unassembled sequences from the specimen. In the case of an assembled dataset, and consensus or representative sequence, this data may be utilized along with the number of reads integrated into each assembled TC consensus. A chimera check algorithm may be utilized such as B2C2. The resulting TC FASTA for each specimen may then be analyzed using a method to evaluate the specimen-derived sequences against existing sequence information from a database or alignment. As an example, sequences derived from the specimen as part of the Level II analysis, may be evaluated against an NCBI database containing all microbial genetic information, or curated databases that contain only specific types of information, such as all 16S rRNA sequences that are considered good quality. The specimen-derived sequences may be compared using a software algorithm such as BLASTn (Altschul et al., 1990, J. Mol. Biol. 215: 403-410) against a database containing known sequence and taxonomic information, for example a database derived or obtained from GenBank (http://ncbi.nlm.nih.gov). For the example, the sequences contained within the curated 16S rRNA database were both >1200 bp and considered of high quality. Scoring criteria may be used as part of the bioinformatics or computational process to evaluate each sequence identity. By example, a post processing algorithm generated best-hit files e.g. those with E-values <10e-114 and bit scores >400 required pathogen identifications. Other algorithms are then utilized to further evaluate the microbial ecology of the specimen. For example, following best-hit processing a secondary post-processing algorithm was utilized to combine genus designations generating a list of taxonomic IDs and their relative predicted abundance within the given specimen. This data may be compiled at any taxonomic level including kingdom, phylum, class, family, group, subgroup, subclass, genus or species.

This taxonomic information is then processed to characterize the ecology of the specimen and may be utilized to derive physiological properties of the organisms present, their antibiotic resistance and/or susceptibilities, and may subsequently be utilized to define treatments, enhancements, or other refinements to enhance or eliminate specific populations or all populations present from the source. This process is performed by utilizing taxonomic, or other information derived from both Level I and Level II assays, executed independently or preferably in combination.

A compilation or analysis or diagnostic report may be generated containing information about the specimen and potential therapeutic strategies from the methods described above. Therapeutics may refer to enhancement or elimination of specific populations, enhancement or elimination of all microbial populations, enhancement or elimination of subsets of the microbial populations from the source of the analyzed specimen.

### Methodology

16S rRNA gene fragments were phylogenetically assigned according to their best matches to sequences based upon BLASTn using WND-BLAST (Dowd et al., 2005, BMC Bioinformatics 6: 93) and a curated database derived from high quality 16S rRNA sequences obtained from RDPII database (Cole et al., 2007, Nucl. Acids Res. 35: D169-D172). Phylogenetic assignments were also evaluated using the Nearest Alignment Space Termination (NAST) database (DeSantis et al., 2006, Nucl. Acids Res. 34: W394-W399). Multiple sequence alignment was done using MUSCLE (with parameter -maxiters 1, -diags1 and -sv) (Edgar, 2004, Nucl. Acids Res. 32: 1792-1797), Clustal W, or a sequence assembly algorithm including the examples of NGEN (DNAstar), PCAP, CLC-bio next generation assembler. Based on the alignments and assemblies, a distance matrix was constructed using DNAdist from PHYLIP version 3.6 with default parameters from Felsenstein (1989, Cladistics 5: 164-166; 2005). These pairwise distances served as input to DOTUR (Schloss & Handelsman, 2005, Appl. Environ. Microbiol. 71: 1501-1506) for clustering the sequences into OTUs of defined sequence similarity that ranged from 0% to 20% dissimilarity. A dissimilarity of 0%-1% in sequences generally provides dramatic overestimation of the species present in a specimen, based upon rarefaction. At 3%-50% dissimilarity, accurate estimation of genera in a specimen is feasible. Thus, at about 3%-10% dissimilarity clinically sufficient accurate estimation of the majority of species present in a given specimen is generated. In this specific example, the clusters based upon dissimilarity of 3%, served as OTUs for generating predictive rarefaction models and for making calculations with the richness (diversity) indexes Ace and Chao1 (Chao & Bunge, 2002, Biometrics 58: 531-539) in Qiime, Pangea, DOTUR, or MoTHUR. These programs may be run on a Microsoft Windows operating system, or any other commercial or custom operating system including Linux or Mac. Data may be processed using a computer. Reports may be stored on a non-transient, computer-readable medium (e.g., RAM or disk); they may be shown on a screen display or printed.

It must be acknowledged that while our methods of amplification and analyses (high annealing temperatures after initial PCR cycles, longer extension times, minimizing as much as possible the number of PCR cycles, and excluding TC with less than 3-fold coverage) attempt to reduce chimera effects on data analysis, a minor population of chimeras might still be expected to be present in this data. Therefore, bioinformatics efforts may be utilized to guide of computational approaches for chimera detection bias in datasets of this magnitude. As used in practice, the potential for overestimation of the maximum predicted OTU is relatively consistent among all specimens yielding clinically relevant comparisons of results.

### Statistical Analysis

Least significant differences (LSDs) were calculated with SAS (version 9.1.3) to compare sample characteristics such as pH, total C, total N, MBC, inflammatory markers (e.g., cytokines and metalloproteases) antibiotic resistance factors, and other diagnostic information not obtained as part of Level I and Level II assays, patient allergy, other patient metadata, comorbidity to define enhancement, or remediation therapies targeted toward a specific specimen or the system from which a specimen is derived. Data generated by these Level I and Level II assays, and the computational or bioinformatics processes may then be utilized to generate databases to further enhance the performance of the software. This involves systematic learning to drive predictive algorithms as part of the methodology.

### Example 2

### Systemic Treatment of Chronic Infection

Chronic wounds represent a significant burden on health care. Decreasing the recovery time can have a significant impact on reducing the costs to treat chronic wounds. In this example, improvement of healing rates in subjects suffering from chronic infections is demonstrated. Our methodology provides the treating clinician the diagnostic information to empower a precise, patient-specific and targeted therapeutic approach resulting in dramatic and unanticipated improved patient outcomes. It should be noted that the prior art, including best-practice literature, does not support universal employment of antibiotic and antibiofilm treatments due in part to the poor comprehensive accuracy of traditional culture-based methods. The clinician is provided with an objective and accurate tool that links accurate microbial detection to bioinformatically derived, comprehensive treatment solutions not previously available, empowering antibiofilm and antimicrobial treatments, including antibiotics and antifungal agents in a universal strategy.

The analysis period in this example was chosen to give a seven-month block for admission, treatment and analysis. Western Institutional Review Board reviewed the proposed study and approved the design and patient safeguards (IRB number 20100213). Data were then populated for each patient identified in each group. For this period, 503 patients were admitted with a chronic wound to Treatment Group A; whereas, 479 patients were identified and admitted to Treatment Group B.

Universal treatments: All patients were managed with standard of care treatments including reperfusion, nutritional support, offloading, compression and management of systemic disease. In addition, patients were managed with a proven clinical regime known in the art as “biofilm-based wound care” which included frequent debridement, biofilm suppression with selective biocides and antibiofilm agents. This algorithm was unchanged and in general use for each patient in both populations.
Treatment Group A: Patients in this group were treated using the universal treatments above. Further, diagnosis of microbial contribution to non-healing was performed using traditional culture based techniques by an independent laboratory to direct antimicrobial pharmacotherapy. Treatment Group B: As in the previous treatment group, patients in this group B were treated using the universal treatments above. Here, diagnosis of the microbial contribution to non-healing was performed using the methodology disclosed herein and pharmacotherapy treatment options were further identified utilizing bioinformatics. Hence, the only difference between the two groups under study was Level I and Level II testing for diagnostic and treatment purposes to address the microbial contribution to non-healing.
Results: 48.5% of patients within Treatment Group A healed completely within six months. By contrast, sixty-two point four (62.4%) of patients within Treatment Group B healed within the same time period (Fisher's exact test, p<0.001; OR=1.76, 95% C.I=1.36 to 2.29). Thus, a significantly higher percentage of patients healed within an equal period of time in Treatment Group B. The definition of “healed” for this example is a fully epithelialized wound, a more burdensome outcome that typically used in the art (e.g., wound size). Furthermore, based upon survival analysis, after controlling for potential confounding factors, the time to heal was significantly shorter in Treatment Group A (p<0.05). Specifically, wound care in Treatment Group B resulted in a 21%, 23%, 25%, and 22% reduction in the time to heal for venous leg ulcers, decubitus ulcers and diabetic foot ulcers and all wounds combined (respectively). It is interesting to note in Treatment Group A, which utilized traditional culture-based microbial diagnostic tools, twenty-three 23% showed no growth or negative results, which provided no pharmacotherapy directions, regardless of any consideration to the accuracy of a positive result. In contrast, for Treatment Group B, which utilized the diagnostic tools of Level I and Level II testing, no clinical specimens were analyzed that produced a negative report, resulting in pharmacotherapy directives for all patients. When compared to other treatment options, standard in the art, these results are quite dramatic and unexpected. Objective study of a significant patient population could confirms the effectiveness of using Level I and Level II diagnosis of these infections in combination with bioinformatically guided patient specific systemic therapy. Use of such pharmacotherapy was not supported by the prior art as a universal strategy. In addition, the use of expensive first-line antibiotics also declined in Treatment Group A resulting in a lower pharmacotherapy cost for those patients. The reduction in associated medical costs along with both humane and ethical considerations associated with such a decrease in overall healing rate highlight the value and utility of providing a new solution to the problem of treating infection. Advances in topical patient-specific therapeutics, directed by Level I and Level II diagnosis, were not a part of this example, but will be reported separately.

TABLE 1
Demographic information for patients in both treatment groups.

| | | Treatment | | Treatment | |
|---|---|---|---|---|---|
| | | Group A | | Group B | |
| Demographics | Number of | 503 | | 479 | |
| | Patients | | | | |
| | Hispanic | 113 | 46.3% | 118 | 39.5% |
| | Black | 34 | 13.9% | 54 | 18.1% |
| | White | 84 | 34.4% | 111 | 37.1% |
| | Other | 8 | 4.9% | 11 | 5.0% |
| | Female | 106 | 44% | 166 | 56% |
| | Male | 137 | 56% | 132 | 44% |
| | Age | Range | Avg. | Range | Avg. |
| | | 5-97 | 61.9 | 2-97 | 59.4 |
| | Diabetes | 98 | 40.2% | 122 | 40.8% |
| Comorbidity | Heart Disease | 56 | 23.0% | 55 | 18.4% |
| | | 40 | 16.4% | 33 | 11.0% |
| | Spinal Cord | 25 | 10.2% | 11 | 3.7% |
| | Impairment | | | | |
| | Immune | 4 | 1.6% | 6 | 2.0% |
| | Suppression | | | | |

TABLE 2
Treatment Group A - Microorganisms Diagnosed with Culturing.

| Microorganisms | #Patients |
|---|---|
| No growth | 15 |
| Staphylococcus aureus (MRSA) | 10 |
| Group D Enterococcus | 8 |
| Coagulase-negative Staphylococcus | 6 |
| Group B Streptococcus | 5 |
| Serratia marcescens | 5 |
| Proteus mirabilis | 3 |
| Pseudomonas spp. | 3 |
| Escherichia coli | 2 |
| Klebsiella pneumoniae | 2 |
| Yeast (not identified) | 2 |
| Bacillus spp. | 1 |
| Morganella morganii | 1 |
| Streptococcus viridans | 1 |
| Kluyvera spp. | 1 |

Clinical microbiology culture results obtained for Treatment Group A.
The species was not identified for “spp.” entries.
“No growth” indicates the culture diagnostic returned a negative result.

TABLE 3
Treatment Group B - Microorganisms Diagnosed with out Culturing.

| Bacterial species | # Patients |
|---|---|
| Finegoldia magna* | 75 |
| Pseudomonas aeruginosa | 74 |
| Staphylococcus aureus | 73 |
| Staphylococcus epidermidis | 71 |
| Anaerococcus vaginalis* | 45 |
| Corynebacterium striatum | 36 |
| Enterococcus faecalis | 36 |
| Serratia marcescens | 34 |
| Anaerococcus lactolyticus* | 33 |
| Propionibacterium acnes* | 28 |
| C. tuberculostearicum* | 27 |
| Pelomonas saccharophila* | 26 |
| Peptoniphilus indolicus* | 24 |
| Streptococcus agalactiae | 23 |
| Escherichia coli | 19 |
| Peptoniphilus ivorii* | 19 |
| Anaerococcus octavius* | 17 |
| Ralstonia pickettii* | 17 |
| Streptococcus mitis | 17 |
| Porphyromonas somerae* | 16 |
| Anaerococcus prevotii* | 14 |
| Peptoniphilus harei* | 13 |
| Anaerococcus hydrogenalis* | 12 |
| Corynebacterium xerosis | 12 |
| Pseudomonas hibiscicola | 12 |
| Ruminococcus obeum* | 12 |
| Staphylococcus haemolyticus | 12 |
| Stenotrophomonas maltophilia | 12 |
| Candidatus Peptoniphilus* | 11 |
| Clostridium hiranonis* | 11 |
| Fusobacterium nucleatum* | 11 |
| Parvimonas micra* | 11 |
| Prevotella buccalis* | 11 |
| S. piscifermentans* | 11 |
| Terrimonas ferruginea* | 11 |
| Burkholderia ambifaria* | 10 |
| Corynebacterium jeikeium | 10 |
| Peptoniphilus lacrimalis* | 10 |
| Staphylococcus capitis | 10 |
| Staphylococcus hominis | 10 |
| Prevotella melaninogenica* | 9 |
| Acinetobacter baumannii | 9 |
| Staphylococcus caprae | 9 |
| Bacteroides fragilis* | 8 |
| C. aurimucosum* | 8 |
| Porphyromonas levii* | 8 |
| Prevotella bivia* | 8 |
| Acinetobacter junii | 7 |
| Bacteroides thetaiotaomicron* | 7 |
| Candida albicans* | 7 |
| Staphylococcus lugdunensis | 7 |
| Streptococcus parasanguinis | 7 |
| Streptococcus sanguinis | 7 |
| Streptococcus thermophilus | 7 |
| Veillonella parvula* | 7 |
| Actinomyces europaeus* | 6 |

Results of comprehensive molecular diagnostics.
Only the top 56 microorganisms were reported out of 584 different microorganisms identified.
Over half of the organisms within this table are difficult to culture, or nearly impossible to diagnose as part of common clinical diagnostic procedures.
The highly fastidious bacteria are indicated by “*”.

### Example 3

### Topical Treatment of Chronic Infection

Topical antibiotics are routinely discouraged in various chronic infections. This paradigm is supported by guidelines published by the CDC, which are subject to interpretation. This paradigm has evolved in modern medicine even though the efficacy of topical antibiotics has never been disproven by objective studies. This paradigm has evolved, at least in part, by the lack of microbial diagnostic tools to objectively and comprehensively determine when topical (or any) antibiotic is appropriate. We sought to, in part, change this paradigm. The most readily available and mature tools for targeting specific bacteria are antibiotics. Systemic concentrations of antimicrobials are limited by systemic toxicity. Further, the microbial biofilms, which populate chronic infections, are known in the art to be 100- to 1500-fold more resistant to such agents. However, concentrations of just such a magnitude are readily obtainable topically. Hence, Level I and Level II diagnosis empowers the appropriate use (targeted) of topical antimicrobial agents to chronic infections. Further, the combination of traditional antimicrobials with antibiofilm agents, also directed by Level I and Level II diagnosis, provides a means to increase the efficacy of the antimicrobials appropriate selection from both segments concomitantly.

In this example, Level I and Level II diagnosis impacts healing rate in a patient suffering from chronic infections. The treating clinician is provided the diagnostic information to empower a precise, subject-specific and specimen-targeted therapeutic approach resulting in unexpectedly improved patient outcomes. As noted above, the state of art as well as best practice literature, does not support the universal employment of topical antibiotic and antibiofilm treatments. Level I and Level II diagnosis provides the clinician with an objective and accurate tool that links accurate microbial detection to bioinformatically derived, comprehensive topical treatment solutions not previously available, empowering antibiofilm and antimicrobial topical treatments, including antibiotics and antifungal agents in a universal strategy.

### Case A:

A 75-year-old white male with diabetes mellitus, venous disease, arterial disease (TCpO₂ of 35 at the foot), a deformed secondary to severe rheumatoid arthritic foot, and neuropathy presented day 1 with a Charcot foot secondary to neuropathy yielding a round plantar ulcer measuring 4 cm in diameter present for over one year. His podiatrist, performed debridement, offloading and a number of different interventions to try to heal the wound, yet it worsened over time. The patient had been in a wheelchair for three months to further offload the wound.

The patient pressed the treating physician, a recognized expert in wound care, for an expected time to heal. Given his past history and all his medical problems, the physician told the patient that he would expect it to take nine months. Based on his experience to date, the patient was quite satisfied with that prediction. He further stated that he would be happy with any prediction that the wound might heal.

The patient was debrided and started on biofilm-based wound care with dressing changes Monday, Wednesday, and Friday. He returned on day 7, when his diagnostic result, showed S. aureus 32% (ct #25.0) and mecA cassettes supported by significant Brevibacterium and Finegoldia. A patient-specific wound gel was ordered by the treating physician, comprising antibiofilm agents, fusidate, clindamycin, and linezolid (all bioinformatically identified). The topical gelwas begun on day 13. By the following week, the wound demonstrated strong improvements. The thick fibrotic reaction of the wound had totally resolved. By day 19, the wound was almost healed. Light debridement was performed and Apligraf® was applied. By day 24, the wound was healed, about three weeks from presentation.

### Case B:

A pleasant 43-year-old Hispanic construction worker presented to a local hospital with a hot, swollen foot with some drainage between the fourth and fifth toe. The patient was taken to surgery for surgical debridement of the foot. At that time of surgery there was found to be tendon involvement as well as two areas where the metatarsals were exposed with obvious osteomyelitis. Significant arterial disease was noted in the chart due to poor bleeding at the time of surgery.

The patient recounted that he was awoken in the recovery room by several physicians, who briefed him on their findings. The surgical team further informed him that due to those findings, they recommended that he immediately go back into surgery for removal of the right leg. Without any vascular testing, the patient was told that they would try to accomplish an amputation at the below knee site but it could end up being above the knee based on what they found at the time of surgery. The patient refused the recommended amputation and demanded an attempt to heal the wound.

Approximately three days after the surgery, the patient remained in the hospital. He and his wife realized that he was not on antibiotics and there was no local wound care to his foot. It was wrapped in Kerlix® (gauze) with no further wound care provided. They concluded that the medical team was in collusion to allow the wound to deteriorate to such a point that he would have to agree to a major limb amputation. On Jul. 27, 2009, the patient signed himself out of the hospital against medical advice and he presented at the wound treatment clinic as a walk-in.

The patient had full evaluation done of his lower extremity. Vascular testing showed that the patient did have adequate perfusion, in that his TCpO₂ was 42 at the right foot despite significant swelling. The ABI was 1.2 and the Laser Doppler toe pressure was 46 with a monophasic wave. While the patient was undergoing noninvasive vascular testing, the Level I diagnostic testing was executed which included S. agalactiae, S. pyogenes, P. aeruginosa, S. aureus, Serratia, mecA cassette, and vancomycin resistance genes along with a universal 16S rRNA diagnostic. The universal diagnostic showed very heavy bacterial presence at the wound site and 54% of the bacteria was found to be S. agalactiae.

With S. aureus and the mecA cassette excluded, along with Pseudomonas aeruginosa excluded, Invanz® (ertapenem) at 1 gram injected intramuscular daily was initiated. The rapid molecular testing directed and identified the antibiotic used. Without Pseudomonas and all of its resistance factors and without methicillin resistance (either S. aureus or coagulase-negative staphylococcus), a broad-spectrum antibiotic such as ertapenem could be started rather than empiric therapy.

Five days later, a comprehensive evaluation of the wound was obtained using the sequencing aspect of Level II. The patient's wound demonstrated 73% S. agalactiae on this test, 18% Peptostreptococcus and 10% Anaerococcus along with 15 other bacterial species identified. No fungus or yeast was identified. Based on this molecular diagnostic, various topical treatment options were identified. The ultimate option selected by the treating clinician included a Sanguitec® LipoGel® base impregnated with sodium fusidate, linezolid, and clindamycin. The choice of this initial topical and this therapeutic change were based directly on the findings from the sequencing aspect of Level II.

The patient responded very dramatically from Jul. 27, 2009 to Aug. 26, 2009. At that time, he asked to return to work but was advised to stay off work for four more weeks. There was still significant waxy biofilm on the surface of the wound. There was also some increased drainage and the wound was decreasing on its healing trajectory. On Aug. 2, 2009, the patient had repeat molecular diagnostics. At this time, Level I testing showed a universal 16S rRNA suggesting only moderate bacteria and limited presence of mecA cassette of (2%). Given the patient's good perfusion and excellent response to topical therapy, which included linezolid, the topical gel was not altered at this time. Subsequently, the comprehensive molecular aspect (Level II testing) arrived on Sep. 9, 2009, showing that the Streptococcus had been reduced to 16%, but Pelomonas was now the dominant organism at over 32% and Corynebacterium jeikeium had emerged at 12%. The topical gel was reformulated to include two anaerobic medications, metronidazole and clindamycin along with fusidate all in Sanguitec® LipoGel® base. The patient showed significant improvement with this reformulation four weeks later (i.e., eight weeks since his admission) of healing was almost complete. The patient went on to heal by 12 weeks after his initial admission for management of his diabetic foot ulcer with involved tendon and osteomyelitis.

This case demonstrates the utility of comprehensive microbial diagnosis followed by subsequent bioinformatic identification of patient-specific treatment options for the successful in clinical management of chronic infections.

### Case C:

A 53-year-old male with longstanding insulin dependent diabetes mellitus and peripheral neuropathy presented with a severe gangrenous left great toe on Oct. 30, 2009. At that time, vascular studies were done and a tissue specimen was sent off for molecular method analysis as disclosed herein. The left foot showed a TCpO₂ of 38, a perfusion pressure at the ankle of 62 with a monophasic waveform, while the ABI was 1.1, all suggesting sufficient perfusion to heal. The patient was treated empirically at the time with a methylcellulose-based antibiofilm gel. Subsequently, the patient was empirically treated with Cubicin® (daptomycin) at 6 mg/kg on Oct. 30, 2009. On Nov. 1, 2009, Level I molecular results were returned (panel of microbes at that time) and identified very high 16S rRNA reporting (universal bacterial load) (this is the “universal bacteria” primer which gives us Quantitation of total bacterial load), yet all the specific targets (i.e., C. albicans, E. faecalis, E. faecium, P. aeruginosa, S. agalactiae, S. aureus, methicillin resistance, vancomycin resistance, S. marcescens, S. pyogenes, and K. pneumoniae. were negative. Based on this result, the patient was switched to Invanz® (ertapenem) at 1 gram injected intramuscularly daily for 14 days.

The comprehensive Level H molecular results were obtained on Nov. 7, 2009 showed a dominance of Prevotella at 44% followed by Bacteroides at 16% of all bacteria present. Collectively, there was an overwhelming anaerobic contribution to the wound biofilm. The patient was maintained on ertapenem but switched to a patient-specific and DNA level-targeted preparation consisting of Sanguitec® LipoGel® base impregnated with metronidazole, clindamycin, and amikacin.

By Dec. 7, 2009, the patient started showing some areas of new blood vessel formation (granulation tissue) but there remained some tissue dying back at the margin. Further, there the first metatarsal head and the second metatarsal head were partially exposed. The patient continued to show a good wound healing trajectory through March of 2010. Subsequently, the wound became red and macerated with the erythema coursing up onto the dorsum of the patient's foot. A second Level II molecular analysis was ordered to determine if a change in the microbial census had occurred. The Level II results were obtained on Feb. 26, 2010, which demonstrated that Pseudomonas had now emerged as dominant at 88% of the microbial population. The minor populations included coagulase-negative staphylococcus and Clostridial species. Further, it is important to note that the comprehensive Level II evaluation also showed Candida parapsilosis in significant abundance. Guided by the bioinformatic options generated by the Level II report, the treating clinician ordered a second patient-specific and DNA level-targeted preparation consisting of Sanguitec® LipoGel® base impregnated with vancomycin, colistin, as well as ketoconazole to address the Candida. The patient responded expediently and went on to heal his wound by May of 2010.

This patient's case history demonstrates that the initial selection of the antibiotics was changed within 24 hours because of superior diagnostic information. By realizing there was no S. aureus present and no mecA resistance information, the treating clinician could expediently change the pharmacotherapeutic strategy from a very expensive systemic antibiotic (daptomycin) to a much more outpatient-friendly daily IM injection of ertapenem which more appropriately targeted the anaerobic contribution to the bioburden of the tissues.

The original suppression of the anaerobic contribution promoted rapid progression prior to the secondary emergence of Pseudomonas and Candida. Further, topical treatments, directed by the second Level II comprehensive analysis, addressed the new contribution of Candida, absent at the beginning of therapy and unidentifiable by traditional culture-based diagnostics. Efficient analysis with DNA level certainty provided by this second Level II evaluation, including the bioinformatic identification of treatment options, allowed the treating clinician to address the change in microbial contribution to resume the patient on a healing trajectory that resulted in the final closure of this limb-threatening wound.

### Clinical Study Utilizing Topical Therapy

In alignment with the systemic treatment study disclosed in Example 2, two treatment groups are currently under a study protocol to assess the impact of targeted topical therapy empowered by Level I and Level II diagnosis compared to culturing techniques. The study had not concluded when this PCT application was filed. Therefore the results are preliminary, but no less noteworthy.

Treatment Group A: Patients in this group were treated using the universal treatments described in Example 2. Additionally, diagnosis of microbial contribution to non-healing was performed using traditional culture-based techniques by an independent laboratory to direct independent antimicrobial pharmacotherapy.

Treatment Group B: As in the previous group, patients in this group were treated using the universal treatments described in Example 2. However, diagnosis of microbial contribution to non-healing was performed using Level I and Level II diagnosis and topical treatment options were further identified utilizing bioinformatics. Hence, the only difference between the two groups under study is the use of Level I and Level II diagnosis prior to treatment for identification of the microbial contribution to non-healing.

Results: As in Example 2, the analysis period in this example was chosen to give a seven-month block for admission, treatment and analysis. Enrollment activities for ˜500 patients were concluded around the end of the third month. At the end of the third month, approximately 50% of patients within Treatment Group B had already completely healed, in contrast to Example 2 where the treatment group that did not benefit from Level I and Level II diagnostic testing (Group A, Example 2), took 6 months to achieve similar healing rates (48.5%). One would project that the ultimate healing percentage for Treatment Group B will be significantly higher than the rate at the end of the third month, given four additional months of treatment (seven month study); however, by the third month, Treatment Group B has already demonstrated a similar healing percentage to Treatment Group A in less than half the treatment time. Thus, patients in Treatment Group B of this molecular diagnostic guided topical therapy group have healed dramatically faster than the control treatment group (Treatment Group A). The definition of “healed” for this example is a fully epithelialized wound, a more burdensome outcome that typically used in the art (e.g., wound size). When compared to other treatment options, standard in the art, the results to date are quite dramatic and unanticipated, especially when one considers the current art does not support the use of topical antibiotics and antibiofilm agents universally. The reduction in associated medical costs along with both humane and ethical considerations associated with such a decrease in overall healing rate highlight the value and utility of Level I and Level II diagnosis in combination with bioinformatically guided patient specific topical therapy.

### Example 4

### Microbial Surveys of Specific Disease States

The Level I embodiment disclosed herein relies in part upon selection of primers to empower this embodiment to report the most targeted and clinically relevant results. As specimens collected from different types of pathological conditions have significantly different microbial frequencies of propagation, microbial surveys were conducted to provide objective direction to the primer selection for Level I. While primer selection for Level I could be “best guess” in the past, the surveys conducted herein and empowered by the Level II embodiment herein, provide the significant advantage to design panels specific to the pathological condition of interest thereby making the panel more targeted and more complete. Such design can rely in part on other factors such as, for example, pathological urgency; however, logic driven selection computations including frequency of identification, frequency weighted for abundance, and abundance weighted for frequency provides significant advantages including accuracy of diagnosis, substantial development costs, and clinical relevance.

### Sequencing Survey of Diabetic Foot Ulcers

| Genera | Samples | Avg % | Std Dev | Min-max % |
|---|---|---|---|---|
| Corynebacterium spp. | 30 | 14.4 | 27.5 | 0.22-80.6 |
| Bacteroides spp. | 25 | 24.2 | 34.8 | 0.15-98.8 |
| Peptoniphilus spp. | 25 | 13.6 | 9.9 | 0.22-38.4 |
| Finegoldia spp. | 23 | 6.7 | 4.1 | 0.65-20.5 |
| Anaerococcus spp. | 22 | 7.7 | 6.1 | 1.28-23.8 |
| Streptococcus spp. | 21 | 36.5 | 26.2 | 1.68-88.8 |
| Serratia spp. | 17 | 21.4 | 22.9 | 0.82-98.4 |
| Unknown-b | 15 | 16.8 | 13.2 | 0.93-62.2 |
| Staphylococcus spp. | 13 | 8.3 | 10.0 | 0.65-32.6 |
| Prevotella spp. | 12 | 7.4 | 24.9 | 0.87-37.3 |
| Peptostreptococcus spp. | 11 | 8.7 | 4.5 | 0.85-41.5 |
| Porphyromonas spp. | 10 | 7.0 | 3.6 | 2.38-24.3 |
| Enterococcus spp. | 10 | 2.8 | 1.2 | 0.31-8.4 |
| Actinomyces spp. | 9 | 5.7 | 5.6 | 1.81-20.2 |
| Pseudomonas spp. | 8 | 14.5 | 11.6 | 0.67-94.3 |
| Clostridium spp. | 8 | 2.3 | 3.2 | 0.75-5.9 |
| Helcococcus spp. | 5 | 2.5 | 3.0 | 0.91-7.3 |
| Brevibacterium spp. | 5 | 1.8 | 0.7 | 0.71-2.46 |
| Varibaculum spp. | 4 | 9.0 | 10.5 | 1.46-27.8 |
| Aerococcus spp. | 4 | 3.0 | 3.6 | 0.47-7.0 |
| Fusobacterium spp. | 3 | 5.6 | 2.6 | 1.99-7.9 |
| Arthrobacter spp. | 3 | 3.8 | 2.5 | 1.85-7.4 |
| Bacillus spp. | 3 | 3.5 | 3.0 | 0.19-7.5 |
| Anaerobiospirillum spp. | 3 | 2.3 | 1.1 | 0.37-3.8 |
| Actinobaculum spp. | 3 | 1.9 | 1.0 | 0.53-2.9 |
| Dermabacter spp. | 3 | 1.6 | 0.9 | 0.78-2.87 |
| Salmonella spp. | 3 | 1.5 | 1.1 | 0.52-3.02 |
| Veillonella spp. | 3 | 1.3 | 4.5 | 1.12-1.49 |
| Citrobacter spp. | 2 | 9.5 | 2.5 | 7.0-12.0 |
| Rothia spp. | 2 | 5.8 | 3.2 | 1.27-10.2 |
| Tissierella spp. | 2 | 4.0 | 2.7 | 1.34-6.6 |
| Propionibacterium spp. | 2 | 3.3 | 0.4 | 2.82-3.7 |
| Proteus spp. | 2 | 3.1 | 2.2 | 0.89-5.3 |
| Aerosphaera spp. | 2 | 2.8 | 1.9 | 1.11-4.5 |
| Peptococcus spp. | 2 | 2.5 | 0.8 | 1.64-3.3 |
| Dermabacter spp. | 2 | 1.2 | 0.7 | 0.61-1.85 |
| Granulicatella spp. | 2 | 1.2 | 0.3 | 0.86-1.51 |
| Brevundimonas spp. | 2 | 0.9 | 0.2 | 0.63-1.07 |

### Sequencing Survey of Venous Leg Ulcers

| | | Frequency | Std | Min | Max |
|---|---|---|---|---|---|
| Pathogen Identified | Count | % | Dev | % | % |
| Bacteroidales A | 22 | 28.2 | 34.8 | 0.1 | 98.1 |
| Staphylococcus aureus | 19 | 41.5 | 37.0 | 0.2 | 97.4 |
| Finegoldia magna | 14 | 12.3 | 26.8 | <0.1 | 80.0 |
| Serratia marcescens | 12 | 43.0 | 42.6 | 0.1 | 99.0 |
| Staphylococcus aureus | 12 | 0.4 | 0.4 | <0.1 | 1.1 |
| Corynebacterium spp. | 11 | 22.7 | 26.8 | 0.1 | 90.2 |
| Peptoniphilus harei | 11 | 16.9 | 26.1 | <0.1 | 82.0 |
| Escherichia coli | 8 | 6.9 | 9.4 | 0.1 | 26.0 |
| Anaerococcus prevotii | 8 | 4.1 | 7.4 | 0.1 | 22.2 |
| Pseudomonas aeruginosa | 7 | 19.4 | 30.7 | 0.1 | 86.7 |
| Staphylococcus spp. | 7 | 2.0 | 4.5 | 0.1 | 12.1 |
| Propionibacterium acnes | 7 | 1.1 | 1.5 | 0.1 | 4.4 |
| Staphylococcus auricularis | 6 | 3.1 | 7.1 | 0.1 | 17.5 |
| Prevotella bryantii | 6 | 1.1 | 1.1 | 0.1 | 3.1 |
| Anaerococcus vaginalis | 5 | 2.7 | 3.2 | 0.2 | 6.7 |
| Corynebacterium spp. | 4 | 10.5 | 11.7 | 0.2 | 26.1 |
| Staphylococcus haemolyticus | 4 | 8.2 | 8.6 | 0.4 | 16.7 |
| Bacteroidales B | 4 | 2.8 | 3.8 | 0.2 | 8.5 |
| Staphylococcus capitis | 4 | 0.4 | 0.4 | 0.1 | 1.0 |
| Streptococcus agalactiae | 3 | 48.2 | 42.2 | 0.2 | 79.6 |
| Porphyromonas somerae | 3 | 7.8 | 11.8 | 0.3 | 21.5 |
| Streptococcus agalactiae | 3 | 6.6 | 5.2 | 0.6 | 9.8 |
| Prevotella marshii | 3 | 1.7 | 2.5 | 0.1 | 4.5 |
| Streptococcus spp. | 3 | 1.5 | 2.5 | <0.1 | 4.3 |
| Actinomyces europaeus | 3 | 0.7 | 0.8 | 0.1 | 1.6 |

### Sequencing Survey of Decubitus Ulcers

| Genera | No. of specimens | Avg % | Std Dev | Max % |
|---|---|---|---|---|
| Streptococcus | 47 | 19.7 | 17.8 | 97.4 |
| Corynebacterium | 45 | 24.6 | 21.3 | 99.3 |
| Staphylococcus | 41 | 12.0 | 9.5 | 99.9 |
| Finegoldia | 32 | 7.5 | 4.6 | 84.2 |
| Pseudomonas | 29 | 13.4 | 7.5 | 81.9 |
| Anaerococcus | 29 | 6.6 | 3.7 | 36.4 |
| Peptoniphilus | 27 | 3.4 | 1.8 | 20.5 |
| Enterococcus | 26 | 8.8 | 3.9 | 79.9 |
| Prevotella | 26 | 7.2 | 3.2 | 68.5 |
| Pelomonas | 19 | 1.7 | 0.6 | 11.1 |
| Clostridium | 18 | 1.8 | 0.6 | 15.9 |
| Bacteroides | 17 | 7.5 | 2.4 | 99.9 |
| Ralstonia | 17 | 1.0 | 0.3 | 4.7 |
| Flavobacterium | 17 | 2.8 | 0.9 | 30.8 |
| Porphyromonas | 16 | 3.4 | 1.0 | 23.5 |
| Serratia | 15 | 20.8 | 6.0 | 91.9 |
| Brevibacterium | 14 | 2.3 | 0.6 | 11.1 |
| Helococcus | 14 | 1.5 | 0.4 | 9.8 |
| Eubacterium | 14 | 0.4 | 0.1 | 2.1 |
| Arthrobacter | 13 | 0.3 | 0.1 | 1.0 |
| Peptostreptococcus | 12 | 2.5 | 0.6 | 8.7 |
| Escherichia | 12 | 3.1 | 0.7 | 12.1 |
| Fusobacterium | 11 | 9.7 | 2.1 | 63.8 |
| Dermabacter | 11 | 0.3 | 0.1 | 0.9 |
| Sulfurospirillum | 10 | 1.0 | 0.2 | 6.3 |

The number of specimens in which each bacterium was identified is provided along with the average percent (Avg %) among the positive specimens, the standard deviation (Std Dev) and the range of percentages among the positive specimens.

### Survey of Spirochetes

Anaplasma phagocytophila
Bartonella henselae**
Borrelia afzelii
Borrelia burgdorferi***
Borrelia garinii
Borrelia hermsii
Borrelia lonestari
Borrelia parkeri
Brachyspira aalborgi
Brachyspira hyodysenteriae**
Coxiella burnetii**
Ehrlichia chaffeensis
Ehrlichia ewingii
Francisella tularensis
Leptospira biflexa**
Leptospira borgpetersenii
Leptospira interrogans
Leptospira kirschneri
Leptospira wolbachii
Mycoplasma fermentans**
Mycoplasma hyopharyngis
Ricketsia species (9 species)*
Treponema carateum
Treponema denticola**
Treponema pallidum
Treponema pertenue
Legend:
no asterisk-significant frequency,
*priority + frequent,
**greater priority + frequent,
***greatest priority + frequent

### Survey of Respiratory Specimens

### Non-Viral Sequencing Survey Results

1. Streptococcus pneumoniae

2. Haemophilus influenza

3. Moraxella catarrhalis

4. Staphylococcus aureus

5. Staphylococcus aureus, community acquired MRSA

6. Streptococcus pyogenes

7. Streptococcus mitis

8. Pseudomonas aeruginosa

9. Yeast (majority Candida & Cryptococcus)

10. Candida albicans

11. Staphylococcus epidermidis

12. Staphylococcus heamolyticus

13. Fusobacterium spp.

14. Eikenella corrodens

15. E. coli

16. Klebsiella spp.

17. Aspergillus spp.

18. Haemophilus parainfluenzae

19. Bacteroides fragilis

20. Proprionibacterium spp.

21. Corynebacterium spp.

22. Turicella spp.

23. Enterococcus spp.

24. Achromobacter spp.

25. Citrobacter spp.

26. Serratia spp.

27. Proteus spp.

28. Prevotella spp.

29. Stenotrophomonas spp.

30. Actinomyces spp.

31. Peptostreptococcus spp.

32. Meningococcus spp.

33. Bacillus spp.

34. Mycobacterium tuberculosis

35. Streptococcus algalactiae

36. Streptococcus mutans

37. Porphyromonas gingivalis

38. Streptococcus sanguinis

39. Veillonella spp.

40. Bartonella spp. (henselae most significant)

41. Mycobacterium avium-intracellulare

42. Mycobacterium bovis

43. Mycoplasma pneumoniae

44. Chlamydophila pneumoniae

45. Legionella spp.

46. Enterobacter aerogenes

47. Enterobacter cloacae

48. Borrelia burgdorferi

49. Moraxella canis

50. Burkholderia spp.

51. Eubacterium spp.

52. Treponema spp.

Viral Survey Results

1. Respiratory Syncytial Virus

2. Influenza A

3. Influenza B

4. Parainfluenza (all paramyxoviruses)

5. Rhinovirus (all picornoviruses)

6. Adenovirus

7. Metapneumovirus

8. Echo Virus

9. Coxsackie Virus

10. Herpes Virus

11. Corona Virus

12. Epstein Barr Virus

13. Cytomegalovirus

14. Enterovirus

### Sequencing Survey of Over 1000 Wounds

| Column 1 - First Table | Column 2 - First Table | Column 3 - First Table |
|---|---|---|
| Pseudomonas aeruginosa | Coryn. pseudodiphtheriticum | Pseudomonas hibiscicola |
| Corynebacterium striatum | Propionibacterium acnes | Prevotella oris |
| Staphylococcus aureus | Helicobacter felis | Streptococcus lutetiensis |
| Staphylococcus epidermidis | Peptoniphilus harei | Prevotella bryantii |
| Serratia marcescens | Staphylococcus cohnii | Corynebacterium accolens |
| Enterococcus faecalis | Citrobacter koseri | Streptococcus gallolyticus |
| Streptococcus agalactiae | Terrimonas ferruginea | Neisseria elongata |
| Finegoldia magna | Enterococcus faecium | Anabaena cylindrica |
| Coryn. tuberculostearicum | Candidatus Peptoniphilus | Corynebacterium confusum |
| Anaerococcus vaginalis | Anaerococcus hydrogenalis | Burkholderia cenocepacia |
| Escherichia coli | Streptococcus pneumoniae | Clostridium ramosum |
| Corynebacterium jeikeium | Staphylococcus hominis | Corynebacterium propinquum |
| Pelomonas saccharophila | Candidatus Helicobacter | Granulicatella adiacens |
| Bacteroides fragilis | Staphylococcus delphini | Hydrocarboniphaga effusa |
| Anaerococcus lactolyticus | Peptoniphilus lacrimalis | Raoultella planticola |
| Streptococcus mitis | Parvimonas micra | Corynebacterium urealyticum |
| Corynebacterium xerosis | Clostridium lituseburense | Pseudomonas panacis |
| Fusobacterium nucleatum | Citrobacter murliniae | Corynebacterium coyleae |
| Prevotella bivia | Burkholderia ambifaria | Dermabacter hominis |
| Acinetobacter baumannii | Corynebacterium aurimucosum | Curvibacter gracilis |
| Proteus mirabilis | Fastidiosipila sanguinis | Macrococcus caseolyticus |
| Anaerococcus prevotii | Corynebacterium lipophiloflavum | Streptococcus anginosus |
| Streptococcus dysgalactiae | Flavobacterium succinicans | Lactobacillus crispatus |
| Ralstonia pickettii | Ruminococcus obeum | Arcanobacterium bernardiae |
| Staphylococcus capitis | Helcococcus kunzii | Helcococcus sueciensis |
| Haemophilus influenzae | Enterococcus avium | Allobaculum stercoricanis |
| Corynebacterium simulans | Roseateles depolymerans | Flavobacterium aquatile |
| Staphylococcus caprae | Turicibacter sanguinis | chicken intestinal |
| Peptoniphilus indolicus | Haemophilus parainfluenzae | Acinetobacter lwoffii |
| Staphylococcus lugdunensis | Rhizobium huautlense | Providencia stuartii |
| Peptostreptococcus stomatis | Bacteroides uniformis | Dolosigranulum pigrum |
| Veillonella parvula | Mycoplasma equirhinis | Clostridium celerecrescens |
| Porphyromonas somerae | Conexibacter woesei | Alkalibacterium iburiense |
| Streptococcus parasanguinis | Merismopedia tenuissima | Clostridium septicum |
| Staphylococcus piscifermentans | Staphylococcus pseudintermedius | Sphingopyxis witflariensis |
| Anaerococcus octavius | Bacteroides ureolyticus | Achromobacter xylosoxidans |
| Klebsiella pneumoniae | Salmonella enterica | Prevotella salivae |
| Staphylococcus haemolyticus | Staphylococcus schleiferi | Prevotella shahii |
| Porphyromonas levii | Streptococcus gordonii | Roseburia intestinalis |
| Brevibacterium antiquum | Streptococcus intermedius | Faecalibacterium prausnitzii |
| Peptoniphilus ivorii | Sporanaerobacter acetigenes | Flavobacterium limicola |
| Moraxella canis | Bacteroides thetaiotaomicron | Halomicronema excentricum |
| Enterobacter cloacae | Bacteroides vulgatus | Rothia amarae |
| Prevotella melaninogenica | Streptococcus cristatus | Pseudomonas alcaligenes |
| Stenotrophomonas maltophilia | Streptococcus thermophilus | Bacteroidales oral |
| Staphylococcus simulans | Pseudomonas chlororaphis | Eubacterium siraeum |
| Streptococcus pyogenes | Actinomyces europaeus | Acinetobacter johnsonii |
| Streptococcus constellatus | Ureaplasma urealyticum | Brevibacterium paucivorans |
| Morganella morganii | Streptococcus sanguinis | Paracoccus yeei |
| Clostridium hiranonis | Acinetobacter junii | Comamonas testosteroni |
| Prevotella buccalis | Prevotella disiens | Proteus vulgaris |

*listed in order of frequency

| Column 1 - Second Table | Column 2 - Second Table | Column 3 - Second Table |
|---|---|---|
| Tepidimicrobium ferriphilum | Eubacterium ruminantium | Clostridium oroticum |
| Xylophilus ampelinus | Alistipes onderdonkii | Actinobaculum schaalii |
| Rhodoferax ferrireducens | Flavobacterium johnsoniae | Fusobacterium equinum |
| Pseudomonas lini | Candidatus Planktoluna | Gemella haemolysans |
| Clostridium bolteae | Bulleidia extructa | Cryobacterium psychrophilum |
| Enhydrobacter aerosaccus | Fusobacterium varium | Afipia felis |
| Clostridium beijerinckii | Janthin. agaricidamnosum | Sphingomonas koreensis |
| Facklamia hominis | Afipia broomeae | Pseudonocardia spinosispora |
| Vibrio parahaemolyticus | Pseudonocardia thermophila | Arcicella aquatica |
| Klebsiella granulomatis | Porphyromonas uenonis | Streptococcus australis |
| Brevundimonas diminuta | Prevotella enoeca | Pseudomonas psychrophila |
| Duganella zoogloeoides | Micrococcus lylae | Lactobacillus johnsonii |
| Beijerinckia indica | blackwater bioreactor | Candidatus Reyranella |
| Staphylococcus saprophyticus | Eubacterium saburreum-like | Corynebacterium appendicis |
| Paenibacillus granivorans | Bacteroides plebeius | Corynebacterium auris |
| Streptomyces griseocarneus | Treponema bryantii | Flavobacterium pectinovorum |
| Clostridium aminophilum | Varibaculum cambriense | Gloeotrichia echinulata |
| Klebsiella oxytoca | Burkholderia thailandensis | Pseudomonas trivialis |
| Nocardiopsis xinjiangensis | Bacteroides acidifaciens | Coryn. glucuronolyticum |
| Prevotella corporis | Collinsella intestinalis | Clostridium purinilyticum |
| Pseudomonas putida | Staphylococcus hyicus | Bacillus licheniformis |
| Veillonella dispar | Clostridium orbiscindens | Acholeplasma laidlawii |
| Arthrobacter albus | Staphylococcus warneri | Planctomyces limnophilus |
| Eubacterium rectale | Clostridium nexile | Herbaspirillum putei |
| Prevotella oulorum | Chitinophaga pinensis | Granulicatella elegans |
| Bacteroides stercoris | Gordonia namibiensis | Mitsuaria chitosanitabida |
| Sphingopyxis chilensis | Balneimonas flocculans | Brevibacterium otitidis |
| Acinetobacter ursingii | Bacteroides finegoldii | Lactobacillus acidophilus |
| Ruminococcus torques | Paracoccus denitrificans | Erwinia billingiae |
| Eubacterium hallii | Aerococcus viridans | Bacillus thuringiensis |
| Corynebacterium afermentans | Clostridium xylanolyticum | Helcococcus ovis |
| Lactobacillus salivarius | Akkermansia muciniphila | Clostridium algidixylanolyticum |
| Prevotella pallens | Prevotella denticola | Ornithinimicrobium humiphilum |
| Sphingomonas faeni | Treponema succinifaciens | Ramlibacter tataouinensis |
| Lachnospiraceae oral | Corynebacterium kroppenstedtii | Pseudobutyrivibrio ruminis |
| Clostridium symbiosum | Staphylococcus sciuri | Brevibacterium epidermidis |
| Bacteroides capillosus | Pedobacter africanus | Novo. pentaromativorans |
| Dialister propionicifaciens | Corynebacterium phocae | Corynebacterium riegelii |
| Prevotella veroralis | Parabacteroides distasonis | Dialister pneumosintes |
| Porphyromonas cangingivalis | Candidatus Amoebinatus | Eubacteriaceae oral |
| Delftia acidovorans | Clostridium saccharolyticum | Paracoccus carotinifaciens |
| Leptolyngbya antarctica | Bradyrhizobium japonicum | Oligella urethralis |
| Eubacterium desmolans | Clostridium disporicum | Phenylobacterium immobile |
| Eubacterium yurii | Collinsella aerofaciens | Pseudomonas stutzeri |
| Clostridium xylanovorans | Dialister invisus | Knoellia sinensis |
| Thermo. aotearoense | Allisonella histaminiformans | Facklamia ignava |
| Phyllobacterium trifolii | Acidovorax defluvii | Porphyromonas endodontalis |
| Actinomyces bowdenii | Microbacterium phyllosphaerae | Sporobacter termitidis |
| Dialister micraerophilus | Roseomonas gilardii | Alkaliflexus imshenetskii |
| Janthinobacterium lividum | Arthrobacter psychrolactophilus | Pasteurella canis |
| Haemophilus felis | Flavobacterium psychrophilum | Peptococcus niger |

*listed in order of frequency, continued from previous table

| Column 1 - Third Table | Column 2 - Third Table | Column 3 - Third Table |
|---|---|---|
| Anoxybacillus flavithermus | Arthrobacter pascens | Pectobacterium carotovorum |
| Xanthomonas oryzae | Chryseobacterium daecheongense | Bacteroides ovatus |
| Corynebacterium amycolatum | Jeotgalicoccus halotolerans | Alcaligenes faecalis |
| Vagococcus fluvialis | Megasphaera elsdenii | Sphingomonas aquatilis |
| Catonella morbid | Shigella dysenteriae | Leucobacter aridicollis |
| Herminiimonas fonticola | Hymenobacter roseosalivarius | Herbaspirillum seropedicae |
| Microvirgula aerodenitrificans | Patulibacter minatonensis | Clostridium scindens |
| Oceanobacillus iheyensis | Acidaminococcus fermentans | Acinetobacter calcoaceticus |
| Brevundimonas nasdae | Eubacterium ventriosum | Lysinibacillus sphaericus |
| Bacillus cereus | Planomicrobium chinense | Curtobacterium flaccumfaciens |
| Brachybacterium alimentarium | Lactococcus lactis | Methylobacterium adhaesivum |
| Brachybacterium muris | Curvibacter delicatus | Haemophilus aegyptius |
| Rikenella microfusus | Anaerovorax odorimutans | Rathayibacter caricis |
| Variovorax paradoxus | Pseudonocardia benzenivorans | Dorea formicigenerans |
| Lactobacillus reuteri | Pseudomonas veronii | Aquabacterium parvum |
| Gemella morbillorum | Corynebacterium imitans | Riemerella anatipestifer |
| Eremococcus coleocola | Clostridium methylpentosum | Haematobacter massiliensis |
| Clostridium hylemonae | Nitrosospira multiformis | Acidovorax temperans |
| Tannerella forsythensis | Clostridium viride | Ruminobacter amylophilus |
| Kluyvera intermedia | Leadbetterella byssophila | Streptococcus mutans |
| Ruminococcus bromii | Actinobaculum massiliae | Naxibacter alkalitolerans |
| Blastococcus saxobsidens | Pseudomonas geniculata | Iodobacter fluviatilis |
| Fusobacterium perfoetens | Porphyromonas asaccharolytica | Ruminococcus flavefaciens |
| Fusobacterium canifelinum | Polaromonas aquatica | Kocuria polaris |
| Leptothrix discophora | Sphingobacterium spiritivorum | Planococcus antarcticus |
| Solibacter usitatus | Cupriavidus necator | Actinomyces meyeri |
| Mogibacterium neglectum | Stigonema ocellatum | Deinococcus indicus |
| Methylocaldum tepidum | Micrococcus antarcticus | Succinivibrio dextrinosolvens |
| Corynebacterium singulare | Porphyromonas catoniae | Atopobium fossor |
| Megamonas hypermegale | Herbaspirillum lusitanum | Corynebacterium mucifaciens |
| Eubacterium xylanophilum | Staphylococcus condimenti | Salinicoccus roseus |
| Actinomyces neuii | Mogibacterium vescum | Alistipes finegoldii |
| Burkholderia silvatlantica | Gracilibacter thermotolerans | Clostridium thermocellum |
| Clostridium hathewayi | Catenibacterium mitsuokai | Zimmermannella bifida |
| Massilia timonae | Microvirga subterranea | Succiniclasticum ruminis |
| Streptococcus canis | Methylophilus methylotrophus | Microbacterium barkeri |
| Dyella yeojuensis | Actinomyces viscosus | Neisseria macacae |
| Leptotrichia goodfellowii | Yersinia rohdei | dehydroabietic acid-degrading |
| Bacteroides intestinalis | Clostridium sartagoforme | Jeotgalicoccus pinnipedialis |
| Modestobacter multiseptatus | Tissierella praeacuta | Trichococcus collinsii |
| Microbacterium foliorum | Pseudonocardia sulfidoxydans | Butyrivibrio hungatei |
| Flavobacterium saccharophilum | Neisseria flavescens | Algoriphagus marincola |
| Streptococcus infantis | Eubacterium sulci | Legionella-like amoebal |
| Clostridium bartlettii | Nocardioides jensenii | Staphylococcus auricularis |
| Clostridium clostridioforme | Lactobacillus gasseri | Lactobacillus delbrueckii |
| Lactococcus garvieae | Ruminococcus lactaris | Legionella pneumophila |
| Bacteroides eggerthii | Prevotella bergensis | Acinetobacter radioresistens |
| Rhodopseudomonas palustris | Sporichthya polymorpha | Citricoccus muralis |
| Clostridium herbivorans | Opitutus terrae | Prevotella loescheii |
| Adhaeribacter aquaticus | Ochrobactrum grignonense | Clostridium histolyticum |
| Clostridium leptum | Blastococcus aggregatus | Alysiella filiformis |

*listed in order of frequency, continued from previous table

| Column 1 - Fourth Table | Column 2 - Fourth Table | Column 3 - Fourth Table |
|---|---|---|
| Prevotella intermedia | Leptotrichia trevisanii | Chryseobacterium piscium |
| Clostridium thermosuccinogenes | Eubacterium contortum | Fibrobacter succinogenes |
| Gordonia amicalis | Aeromonas hydrophila | Nocardioides kribbensis |
| Propionibacterium granulosum | Phascolarctobacterium faecium | Sporolactobacillus nakayamae |
| Kocuria kristinae | Fluviicola taffensis | Plantibacter flavus |
| Clostridium sordellii | Streptococcus iniae | Dermocarpella incrassata |
| Pseudoxanthomonas mexicana | Paraliobacillus ryukyuensis | Dyadobacter ginsengisoli |
| Sporosarcina macmurdoensis | Sanguibacter inulinus | Anoxybacillus kestanbolensis |
| Candidatus Cuticobacterium | Anaerofilum agile | Brevibacterium stationis |
| Gracilibacillus halotolerans | Frigoribacterium faeni | Flavobacterium saliperosum |
| Propionibacterium avidum | Atopobium parvulum | Enterococcus raffinosus |
| Methylibium petroleiphilum | Caulobacter vibrioides | Nocardioides aestuarii |
| Hahella ganghwensis | Porphyrobacter donghaensis | Candidatus Nitrotoga |
| Clostridium amygdalinum | Hyphomicrobium facile | Nocardiopsis metallicus |
| Rhodopila globiformis | Hymenobacter rigui | Weeksella virosa |
| Providencia rettgeri | Actinoplanes capillaceus | Peredibacter starrii |
| Fusobacterium necrophorum | Alistipes putredinis | Anaerococcus tetradius |
| Empedobacter brevis | Pedobacter aquatilis | Clostridium pasteurianum |
| Eikelbloom type | Pedomicrobium australicum | Chryseobacterium shigense |
| Acidovorax avenae | Arthrobacter globiformis | Fusibacter paucivorans |
| Bacillus subtilis | Enterococcus devriesei | Clostridium lactatifermentans |
| Arcobacter cryaerophilus | Tuber borchii | Campylobacter gracilis |
| Rhodospirillum rubrum | Selenomonas ruminantium | Acinetobacter haemolyticus |
| Methylobacterium extorquens | Anaerotruncus colihominis | Clostridium citroniae |
| Cronobacter dublinensis | Streptococcus sobrinus | Brachy. paraconglomeratum |
| Bacteroides caccae | Leptotrichia shahii | Corynebacterium variabile |
| Bacteroides nordii | Rhizobium etli | Rubrobacter radiotolerans |
| Bacillus benzoevorans | Methylomicrobium album | Clostridium akagii |
| Ignavigranum ruoffiae | Eubacterium eligens | Sutterella stercoricanis |
| Globicatella sulfidifaciens | Anaerobiospirillum thomasii | Verrucomicrobium spinosum |
| Bacteroides dorei | Prevotella tannerae | Skermanella parooensis |
| Actinomyces radingae | Neisseria polysaccharea | Candidatus Aquirestis |
| Zoogloea oryzae | Tetrasphaera japonica | Bacteroides massiliensis |
| Micrococcus luteus | Planococcus maitriensis | Alvinella pompejana |
| Victivallis vadensis | Prevotella nigrescens | Stenotrophomonas rhizophila |
| Candidatus Nostocoida | Pseudomonas mendocina | Algoriphagus yeomjeoni |
| Candidatus Prevotella | Prevotella marshii | Clostridium rectum |
| Oribacterium sinus | Chitinimonas taiwanensis | Ralstonia insidiosa |
| Lactobacillus animalis | Hydrogenophaga atypica | Aquabacterium commune |
| Denitratisoma oestradiolicum | Enterococcus mundtii | Selenomonas sputigena |
| Candidatus Burkholderia | Nostocoida limicola | Alkaliphilus transvaalensis |
| Pirellula staleyi | Alishewanella fetalis | Gordonia desulfuricans |
| Anaerovibrio lipolyticus | Aeromonas punctata | Rhodoplanes roseus |
| Corynebacterium minutissimum | Clostridium perfringens | Alistipes shahii |
| Flavobacterium frigoris | Carnobacterium pleistocenium | Methylobacterium aquaticum |
| Serratia liquefaciens | Prevotella heparinolytica | Staphylococcus lentus |
| Chryseobacterium wanjuense | Hymenobacter actinosclerus | Pedobacter cryoconitis |
| Microbacterium kitamiense | Prevotella buccae | Propion. lymphophilum |
| Facklamia languida | Lachnospira pectinoschiza | Bacteroides coprocola |
| Mogibacterium pumilum | Sphingopyxis alaskensis | Aerococcus urinae |
| Methylobacterium variabile | Chloroflexus aurantiacus | Polynucleobacter necessarius |

*listed in order of frequency, continued from previous table

| Column 1 - Fifth Table | Column 2 - Fifth Table | Column 3 - Fifth Table |
|---|---|---|
| Candidatus Rhodoluna | Arthrobacter agilis | Virgibacillus necropolis |
| Flavobacterium gelidilacus | Eikenella corrodens | Sporolactobacillus terrae |
| Gillisia mitskevichiae | Sanguibacter suarezii | Serratia proteamaculans |
| Exiguobacterium aurantiacum | Sphingobium japonicum | Enterobacter hormaechei |
| Megasphaera paucivorans | Anaerofilum pentosovorans | Pseudoxanthomonas suwonensis |
| Rubellimicrobium thermophilum | Sneathia sanguinegens | Kocuria palustris |
| Porphyromonas circumdentaria | Microbacterium xylanilyticum | Clostridium ultunense |
| Roseiflexus castenholzii | Clostridium fimetarium | Kineococcus marinus |
| Ureibacillus terrenus | Nocardiopsis halotolerans | Kocuria carniphila |
| Eubacterium tortuosum | Novosphingobium tardaugens | Agrococcus jenensis |
| Arthrobacter nicotianae | Cryptosporangium aurantiacum | Pseudomonas psychrotolerans |
| blood disease | Roseburia cecicola | Brevundimonas kwangchunensis |
| Anaeroplasma abactoclasticum | Bacillus clausii | Legionella taurinensis |
| Microcella alkaliphila | Legionella wadsworthii | Lamellibrachia columna |
| Janibacter melonis | Clostridium colicanis | Flavobacterium xinjiangense |
| Aerococcus sanguinicola | Nevskia ramosa | Eggerthella lenta |
| Methylobacterium populi | Atopostipes suicloacalis | Arthrobacter nicotinovorans |
| Clostridium aerotolerans | Sarcina maxima | Shigella sonnei |
| Acidovorax delafieldii | Bisgaard Taxon | Deinococcus geothermalis |
| Microbacterium thalassium | Lactobacillus aviarius | Flavobacterium psychrolimnae |
| Yaniella halotolerans | Sporomusa aerivorans | Sporosarcina globispora |
| Aquabacterium citratiphilum | Pseudomonas argentinensis | Propionivibrio limicola |
| Citrobacter braakii | Lentzea waywayandensis | Staphylococcus carnosus |
| Clostridium methoxybenzovorans | Arthrobacter arilaitensis | Ochrobactrum anthropi |
| Corynebacterium matruchotii | Proteiniphilum acetatigenes | Belnapia moabensis |
| Solirubrobacter pauli | Conchiformibius steedae | Parabacteroides goldsteinii |
| Mesorhizobium loti | Arthrobacter oxydans | Abiotrophia defectiva |
| Capnocytophaga ochracea | Eubacterium biforme | Bacillus niacini |
| Candidatus Xiphinematobacter | Okibacterium fritillariae | Ottowia thiooxydans |
| Mogibacterium timidum | Geobacillus subterraneus | Flectobacillus major |
| Porphyromonas macacae | Streptococcus peroris | Dietzia natronolimnaea |
| Schlegelella thermodepolymerans | Paenibacillus agaridevorans | Cellvibrio gandavensis |
| Flavobacterium soli | Delflia tsuruhatensis | Clostridium straminisolvens |
| Lysobacter antibioticus | Terrabacter terrae | Cellulosimicrobium cellulans |
| Cellvibrio mixtus | Streptococcus devriesei | Rahnella aquatilis |
| Crypto. minutisporangium | Novosphingobium stygium | Lachnobacterium bovis |
| Enterococcus cecorum | Pseudoxan. broegbernensis | Pediococcus acidilactici |
| Veillonella atypica | Flavobacterium hydatis | Syntrophococcus sucromutans |
| Clostridium sporosphaeroides | Streptomyces luridiscabiei | Cupriavidus metallidurans |
| Gemmatimonas aurantiaca | Staphylococcus arlettae | Novosphingobium lentum |
| Paludibacter propionicigenes | Novosphingobium hassiacum | Aquicella siphonis |
| Clostridium glycolicum | Algoriphagus ornithinivorans | Kocuria rosea |
| Campylobacter rectus | Eubacterium ramulus | Yeosuana aromativorans |
| Niastella koreensis | Bergeyella zoohelcum | Methylocaldum szegediense |
| Clostridium paraputrificum | Eubacterium angustum | Eubacterium infirmum |
| Cetobacterium somerae | Wautersiella falsenii | Prevotella zoogleoformans |
| Actinotalea fermentans | Pseudomonas pseudoalcaligenes | Rothia dentocariosa |
| Clostridium innocuum | Olsenella profusa | Pantoea stewartii |
| Staphylococcus kloosii | Bacillus pumilus | Acetanaerobacterium elongatum |
| Clostridium spiroforme | Anaeroplasma varium | Devosia riboflavina |
| Mycobacterium chelonae | Mycoplasma orale | Staphylococcus pasteuri |

*listed in order of frequency, continued from previous table (subsequent 733 species truncated)

### Other Embodiments

All such variations are intended to be within the scope and spirit of the invention.

Although some embodiments are shown to include certain features, the inventors specifically contemplate that any feature disclosed herein may be used together or in combination with any other feature or any other embodiment of the invention. It is also contemplated that any feature disclosed herein may be specifically excluded from any embodiment of the invention.

The foregoing embodiments demonstrate experiments performed and techniques contemplated by the present inventors in making and carrying out the invention. It is believed that these embodiments include a disclosure of methodologies for analysis and reporting, which serve both to apprise the art of the practice of the invention and to demonstrate its usefulness. It will be appreciated by those of skill in the art that the embodiments disclosed herein are only illustrative and, in general, numerous equivalent methods and techniques may be employed to achieve the same result.

Unless otherwise defined, all technical terms used herein have the same meaning as known by those of skill in the art to which this invention belongs. Although techniques and materials similar or equivalent to those described herein can be used in the practice or testing of the present invention, suitable techniques and materials are described above. All publications, patent applications, patents, and other references mentioned herein are incorporated by reference in their entirety. In the case of conflict, the present specification, including definitions, will control. In addition, the materials, methods, and examples are illustrative only and not intended to be limiting.

1. A method for detecting a plurality of different microorganisms in at least one specimen obtained from a subject, the method comprising in any order:
(a) sequencing a plurality of genetic materials in a specimen; wherein the genetic materials are selected from the group consisting of amplified templates, genomes, or metagenomes; wherein the presence of a sequence indicative of a genus, species, or strain of microorganism is sufficient to identify or quantify proportionally that microorganism among the plurality of different microorganisms in a specimen; and
(b) amplifying target polynucleotides in a specimen to quantify the total or individual number of the plurality of different microorganisms in a specimen.

2. A method for detecting a plurality of different microorganisms in at least one specimen obtained from a subject, the method comprising in any order:
(a) amplifying target polynucleotides in a specimen to produce template nucleic acids; wherein the presence of a template indicative of a genus, species, or strain of microorganism is sufficient to identify or quantify the plurality of different microorganisms in a specimen; and
(b) sequencing a plurality of genetic materials in a specimen; wherein the genetic materials are selected from the group consisting of amplified templates, genomes, or metagenomes; wherein the presence of a sequence indicative of a genus, species, or strain of microorganism is sufficient to identify or quantify proportionally that microorganism among the plurality of different microorganisms in a specimen.

3. The method according to claim 2 further comprising in any order:
(c) amplifying target polynucleotides in a specimen to quantify the total or individual number of the plurality of different microorganisms in a specimen.

4. The method according to claim 1, wherein N different specimens are amplified in parallel reactions by tagging target polynucleotides of a first specimen with a first marker, tagging target polynucleotides of a second specimen with a second marker, and so on mutatis mutandis to tagging target polynucleotides of an Nth specimen with an Nth marker prior to amplifying or sequencing; a marker is found in an amplified or sequenced template nucleic acid; and the marker identifies the template nucleic acid as derived from a particular specimen.

5. The method according to claim 4, wherein at least 10, at least 25, at least 50, at least 75, at least 100, or at least 250 different specimens are amplified and sequenced in parallel reactions.

6. The method according to claim 1, wherein at least one microbial genus, species, and/or strain detected at a proportion less than 1%, less than 2.5%, or less than 5% and/or a number less than 10, less than 100, less than 1000, or less than 10,000 in the specimen is not reported as detected or is reported as not detected.

7. The method according to claim 1, wherein at least five, ten, 15, 20, 25, 30, 35, 40, 45, 50, 100, 1000 or 10,000 different microbial genera, species, and/or strains are detected in a specimen.

8. The method according to claim 1, wherein amplification reactions are performed using a nucleic acid amplifier instrument and/or sequence reactions are performed using a nucleic acid sequencer instrument.

9. A method for detecting a plurality of different microorganisms in at least one wound specimen obtained from a subject intended for medical diagnosis, the method comprising:
(a) amplifying target polynucleotides in a specimen with a set of primer oligonucleotides to produce template nucleic acids, wherein the presence of a template indicative of a specific taxonomic designation of genus is sufficient to identify or quantify that microorganism in a specimen;
(b) wherein the set of primers are specific for detection of Pseudomonas, Corynebacterium, Staphylococcus, Serratia, Enterococcus, Streptococcus, Finegoldia, and Anaerococcus; and
(c) wherein the set of primers are specific for detection of any one set of the following:
(i) Set A: Escherichia, Pelomonas, Bacteroides, Fusobacterium, Prevotella, Acinetobacter, Proteus, and Ralstonia; or
(ii) Set B: Haemophilus, Peptoniphilus, Peptostreptococcus, Veillonella, Porphyromonas, Klebsiella, Brevibacterium, and Moraxella; or
(iii) Set C: Enterobacter, Stenotrophomonas, Morganella, Clostridium, Propionibacterium, Helicobacter, Citrobacter, and Terrimonas; or (iv) Set D: Candidatus, Parvimonas, Burkholderia, Fastidiosipila, Flavobacterium, Ruminococcus, Helcococcus, and Roseateles; or
(v) Set E: Turicibacter, Rhizobium, Mycoplasma, Conexibacter, Merismopedia, Salmonella, Sporanaerobacter, and Actinomyces; or
(vi) Set F: Neisseria, Anabaena, Granulicatella, Hydrocarboniphaga, Raoultella, Dermabacter, Curvibacter, and Macrococcus; or
(vii) Set G: Lactobacillus, Arcanobacterium, Allobaculum, Providencia, Brevibacterium, Alkalibacterium, Eubacterium, and Achromobacter.

10. A method for detecting a plurality of different microorganisms in at least one respiratory specimen obtained from a subject intended for medical diagnosis, the method comprising:
(a) amplifying target polynucleotides in a specimen with a set of primer oligonucleotides to produce template nucleic acids, wherein the presence of a template indicative of a specific taxonomic designation of species is sufficient to identify or quantify that microorganism in a specimen;
(b) wherein the set of primers are specific for detection of Streptococcus pneumoniae, Haemophilus influenza, Moraxella catarrhalis, Staphylococcus aureus, methicillin resistant staphylococcus, Streptococcus pyogenes, Streptococcus mitis, and Pseudomonas aeruginosa; and
(c) wherein the set of primers are specific for detection of any one set of the following:
(i) Set A: Yeast spp., Candida albicans, Staphylococcus epidermidis, Staphylococcus haemolyticus, Fusobacterium spp., Eikenella corrodens, E. coli, and Klebsiella spp.; or
(ii) Set B: Aspergillus spp., Haemophilus parainfluenzae, Bacteroides fragilis, Proprionibacterium spp., Corynebacterium spp., Turicella spp., Enterococcus spp., and Achromobacter spp.; or
(iii) Set C: Citrobacter spp., Serratia spp., Proteus spp., Prevotella spp., Stenotrophomonas spp., Actinomyces spp., Peptostreptococcus spp., and Meningococcus spp.; or
(iv) Set D: Bacillus spp., Mycobacterium tuberculosis, Respiratory Syncytial Virus, Influenza A, Influenza B, Parainfluenza, Rhinovirus, and Adenovirus; or
(v) Set E: Metapneumovirus, Echo Virus, Coxsackie Virus, Herpes Virus, Corona Virus, Epstein Barr Virus, Cytomegalovirus, and Enterovirus; or
(vi) Set F: Streptococcus algalactiae, Streptococcus mutans, Porphyromonas gingivalis, Streptococcus sanguinis, Veillonella spp., Bartonella spp., Mycobacterium avium, Mycobacterium bovis, and Mycoplasma pneumoniae; or
(vii) Set G: Chlamydophila pneumoniae, Legionella spp., Enterobacter aerogenes, Enterobacter cloacae, Borrelia burgdorferi, Moraxella canis, Burkholderia spp., Eubacterium spp., and Treponema spp.

11. A method for detecting a plurality of different microorganisms in at least one blood specimen obtained from a subject intended for medical diagnosis, the method comprising:
(a) amplifying target polynucleotides in a specimen with a set of primer oligonucleotides to produce template nucleic acids, wherein the presence of a template indicative of a specific taxonomic designation of species is sufficient to identify or quantify that microorganism in a specimen;
(b) wherein the set of primers are specific for detection of Borrelia burgdorferi, Bartonella henselae, and Brachyspira hyodysenteriae; and
(c) wherein the set of primers are specific for detection of any one set of the following:
(i) Set A: Coxiella burnetii, Leptospira biflexa, Mycoplasma fermentans, and Mycoplasma hyopharyngis; or
(ii) Set B: any three of Borrelia afzelii, Borrelia garinii, Borrelia hermsii, Borrelia lonestari, and Borrelia parkeri; or
(iii) Set C: Mycoplasma fermentans and Mycoplasma hyopharyngis;
(iv) Set D: any four of Rickettsia rickettsii, Rickettsia akari, Rickettsia conorii, Rickettsia sibirica, Rickettsia australis, Rickettsia japonica, Rickettsia africae, Rickettsia prowazekii, and Rickettsia typhi; or
(v) Set E: any two of Anaplasma phagocytophila, Francisella tularensis, Brachyspira aalborgi, Ehrlichia chaffeensis, and Ehrlichia ewingii; or
(vi) Set F: any two of Leptospira borgpetersenii, Leptospira interrogans, Leptospira kirschneri, and Leptospira wolbachii; or
(vii) Set G: any two of Treponema denticola, Treponema carateum, Treponema pallidum, and Treponema pertenue.

12. The method according to claim 1, wherein at least Candida albicans, extended spectrum beta lactamase resistance, Enterococcus faecalis, Enterococcus faecium, Klebsiella pneumoniae, Staphylococcus agalactiae, Staphylococcus aureus, Staphylococcus marcescens, Staphylococcus pyogenes, coagulase-negative staphylococcus, methicillin-resistant staphylococcus, vancomycin-resistant staphylococcus, Pseudomonas aeruginosa, one or multiple antibiotic-resistant bacterial strains, or a combination thereof are reported as not detected or not detected in the specimen.

13. A method for treating a subject with an infection, the method comprising detecting a plurality of different microorganisms in at least one specimen obtained from the subject according to claim 1, then administering a treatment regimen that is effective against at least one or multiple microorganisms that were detected.

14. The method according to claim 13, wherein at least one or multiple antibiotics, one or more antibiofilm agents, or a combination thereof are administered to the subject.

15. The method according to claim 13, wherein at least one treatment regimen is provided in a report as a part of or within seven days of reporting detection of a plurality of different microorganisms in a specimen.

16. A method for monitoring a subject with an infection, the method comprising detecting a plurality of different microorganisms in at least one specimen obtained from the subject according to claim 1 after initial treatment of the infection.

17-18. (canceled)


SEQUENCE LISTING (2 sequences)

SEQ ID NO: 1 - DNA, 8 bp, Artificial Sequence
  synthetic tag
accgtcat

SEQ ID NO: 2 - DNA, 6 bp, Artificial Sequence
  synthetic tag
agcgtc